(19) United States
(12) Patent Application Publication
(10) Pub. No.: US 2026/0092080 A1
(43) Pub. Date: Apr. 2, 2026

(54) NOVEL RUTHENIUM COMPLEXES, METHOD OF THEIR SYNTHESIS, INTERMEDIATE COMPOUNDS USED IN THIS METHOD, METHOD OF THEIR SYNTHESIS AND USE OF NOVEL RUTHENIUM COMPLEXES IN OLEFIN METATHESIS REACTIONS

(71) Applicant: Uniwersytet Warszawski, Warszawa (PL)

(72) Inventors: Anna KAJETANOWICZ, Warszawa (PL); Adrian SYTNICZUK, Minsk Mazowiecki (PL); Filip STRUZIK, Warszawa (PL)

(73) Assignee: Uniwersytet Warszawski, Warszawa (PL)

(21) Appl. No.: 19/110,135

(22) PCT Filed: Sep. 8, 2023
(86) PCT No.: PCT/IB2023/058921

§ 371 Date: Mar. 10, 2025

(30) Foreign Application Priority Data
Sep. 8, 2022 (PL) PL442226

(51) Int. Cl. C07F 15/00; B01J 31/22; C07C 6/04; C07F 17/02

(52) U.S. Cl. CPC C07F 15/0046; B01J 31/2208; B01J 31/2273; B01J 31/2278; B01J 31/2291; B01J 31/2295; C07C 6/04; C07F 17/02; B01J 2231/543; B01J 2531/004; B01J 2531/0205; B01J 2531/821; B01J 2531/842; C07C 2531/22

(57) ABSTRACT

The object of the invention is a novel ruthenium complex of the general formula Ru-1, in which all the variable substituents have the meanings defined in the description. Also an object of the invention is a method for obtaining the ruthenium complex, a ligand precursor intermediate compound used in the preparation of the ruthenium complex CAAC-1 and the use of this ruthenium complex as a (pre)catalyst in olefin metathesis reactions.

The subject of the invention are novel ruthenium complexes with CAAC type ligands (Cyclic Alkyl Amino Carbene) which have found a widespread use as catalysts and/or (pre)catalysts of olefin metathesis reactions and their use in olefin metathesis reactions. The subject of the invention also includes intermediate compounds used to synthesize novel ruthenium complexes with CAAC ligands, as well as the method for synthesis of novel ruthenium complexes with CAAC ligands. This invention is used as desired tool in the widely understood organic synthesis, in the selective synthesis of olefins with a C═C bond, in particular in cross metathesis reaction with ethylene; ethenolysis.

Significant progress has been made in recent years in the use of olefin metathesis in organic synthesis [R. H. Grubbs (Editor), A. G. Wenzel (Editor), D. J. O'Leary (Editor), E. Khosravi (Editor), Handbook of Olefin Metathesis, 2. Edition, 3 volumes, 2015, Wiley-VCH Verlag GmbH & Co. KGaA, 1608 pages]. Many ruthenium-based homogeneous olefin metathesis catalysts are known in the state-of-the-art which show high activity in various types of metathesis reactions, as well as high tolerance to functional groups present in the substrate/product. Due to the combination of these features, metathesis catalysts are of key importance in modern organic synthesis and in the industry. The most widely present ruthenium complexes in the literature and the ones finding the most widespread use in olefin metathesis reactions include Grubbs-type ruthenium complexes (Gru-I, Gru-II and Gru-III), Hoveyda-Grubbs complexes (Hov-I and Hov-II), as well as indenylidene complexes (Ind-I, Ind-II and Ind-III), 1^{st}, 2^{nd} and 3^{rd} generation [Grubbs et al. Chem. Rev. 2010, 110, 1746-1787; Nolan et al. Chem. Commun. 2014, 50, 10355-10375]. In other cases, most structures of olefin metathesis catalysts are derived from the aforementioned ruthenium complexes.

Recently, the NHC ligand in the ruthenium catalyst structure has been replaced by a cyclic (alkyl) (amino) carbene ligand (CAAC), and resulted complexes occupy an important place in modern organic synthesis due to their use i.e. in cross metathesis reactions and in ring-closing metathesis [Grubbs et al. Chem. Rev., 2010, 110, 1746-1787; WO2017055945A1].

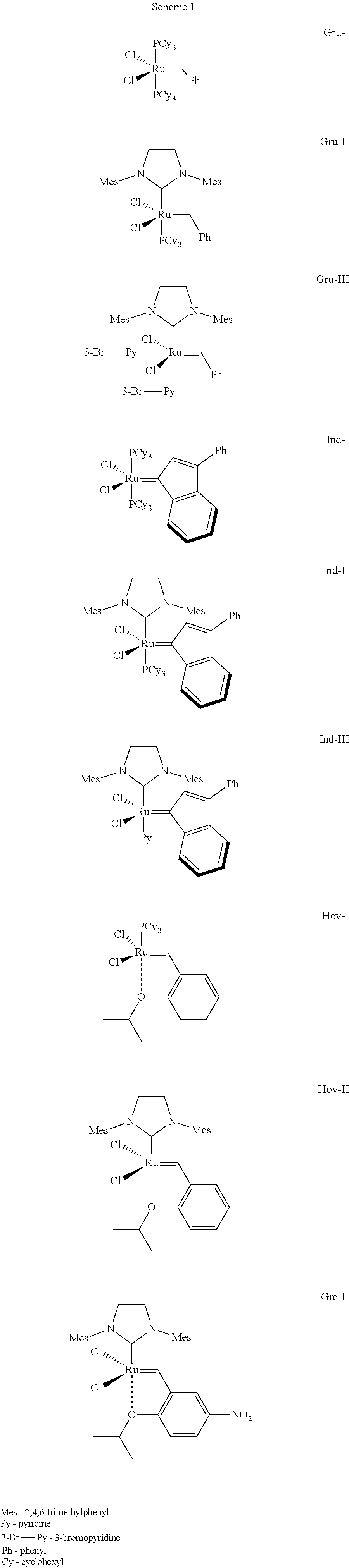

In modern organic synthesis, in an era of increasingly scarce resources, including fossil fuels, and the consequent risk of a shortage of raw materials for the synthesis for example, polymers based on products derived, for example, from crude oil, it is very important to develop new technologies and reactions that enable the synthesis of target compounds. Such processes include the ethenolysis reaction, particularly the ethenolysis of methyl/ethyl derivatives of long-chain fatty acids. In particular, ruthenium complexes with CAAC ligands in the ruthenium coordination sphere are used for this purpose. The first literature report on ruthenium catalysts for olefin metathesis with CAAC ligands dates back to 2005 [Bertrandt et al. Angew. Chem. Int. Ed., 2005, 44, 5705-5709]. In this scientific publication, Bertrandt described for the first time the CAAC ligands and their use in organic synthesis. In a subsequent publication from 2007 [Bertrandt et al. Angew. Chem. Int. Ed., 2007, 46,7262-7265], Bertrandt described first method of the synthesis of ruthenium complexes with CAAC ligands. In both cases, the ligands contained 2,6-diisopropylbenzene at the nitrogen atom, and either two methyl (Ru5) or cyclohexyl substituent (Ru10) at the carbon atom C2. Next, the team of Prof. Bertrand, in cooperation with the team of Prof. Grubbs, synthesized and tested 17 catalysts in terms of their activity in the ethenolysis reaction of methyl oleate, in which the CAAC ligand contained symmetric substituents at the nitrogen atom: mesityl, 2,6-diisopropylbenzene, 2,6-diethylbenzene, as well as asymmetric ones 2-ethyl-6-methylbenzene, 2-isopropyl-6-methylbenzene, 2-methyl-6-tertbutylbenzene, while at the C2 carbon atom, two of the following substituents: methyl, ethyl, n-propyl, cyclohexyl, adamantyl or phenyl [Bertrandt et al. Angew. Chem. Int. Ed., 2015, 54, 1919-1923]. These catalysts showed high activity towards the C—C double bond of methyl oleate in the presence of ethylene overpressure, leading to the formation of valuable industrial products in the olefin metathesis reaction: 1-decene and 9-decenoic acid methyl ester.

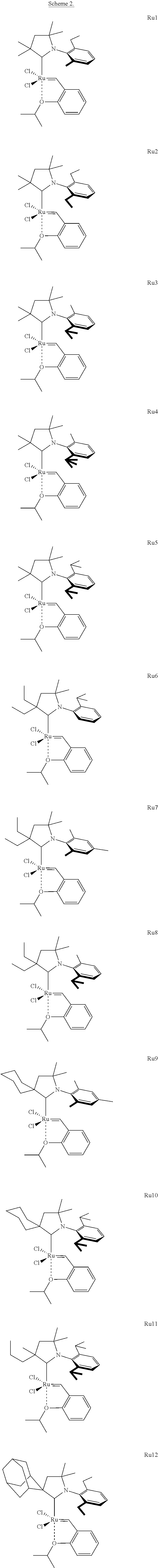

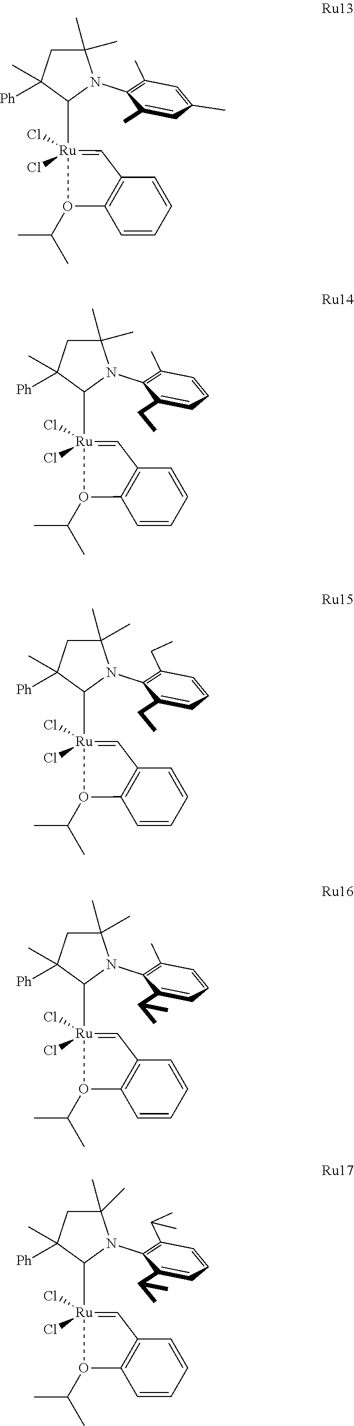

In 2017, Gawin et al. first published a method for a synthesis of indenylidene-type complexes with two CAAC ligands [Gawin et al., Angew. Chem. Int. Ed. 2017, 56, 981-986; EP3356379B1]. The publication presents a method for the preparation of indenylidene complexes with two CAAC ligands, and tests the activity of the complexes in selected reactions including macrocyclisation, ethenolysis, and cross metathesis reaction of α-olefins. This document also discloses a novel approach to the synthesis of Hoveyda-Grubbs catalysts with CAAC ligands, involving thermal dissociation of one CAAC ligand in an indenylidene complex, followed by reaction of the intermediate with the relevant styrene.

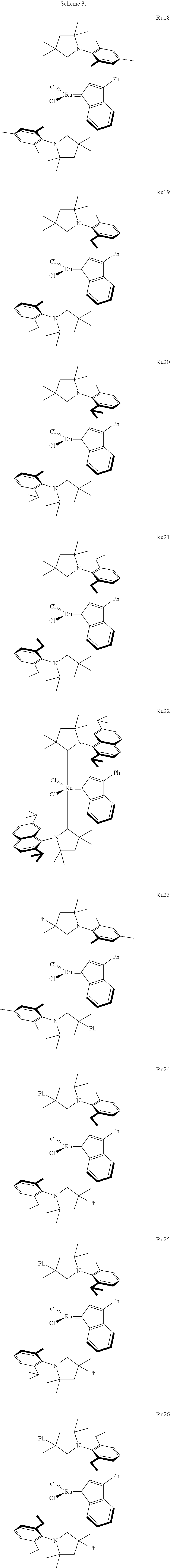

Also in 2017, Gawin et al. published a method to synthesize Hoveyda-Grubbs analogues with a nitro group in the para position as substrates using bis-CAAC complexes [Gawin et al. ACS Catal. 2017, 7, 5443-5449]. The complexes proved to be effective in macrocyclization and cross-metathesis reactions with acrylonitrile.

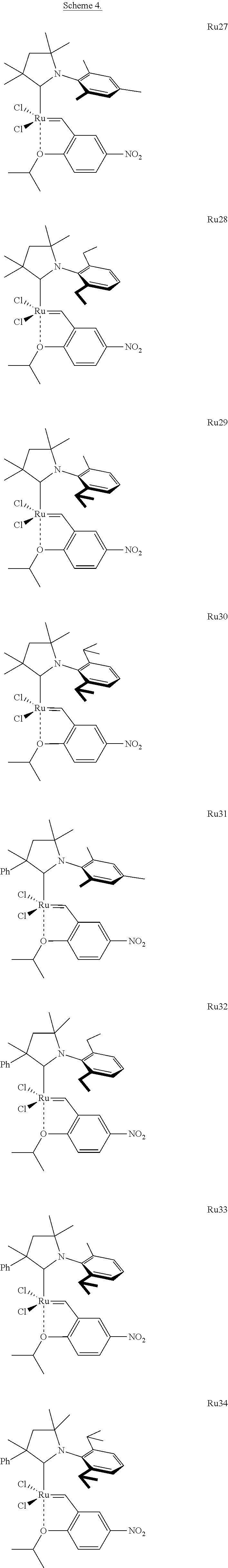

The European patent [EP3356379B1] discloses the structures Ru35-Ru37 with a modified benzylidene fragment.

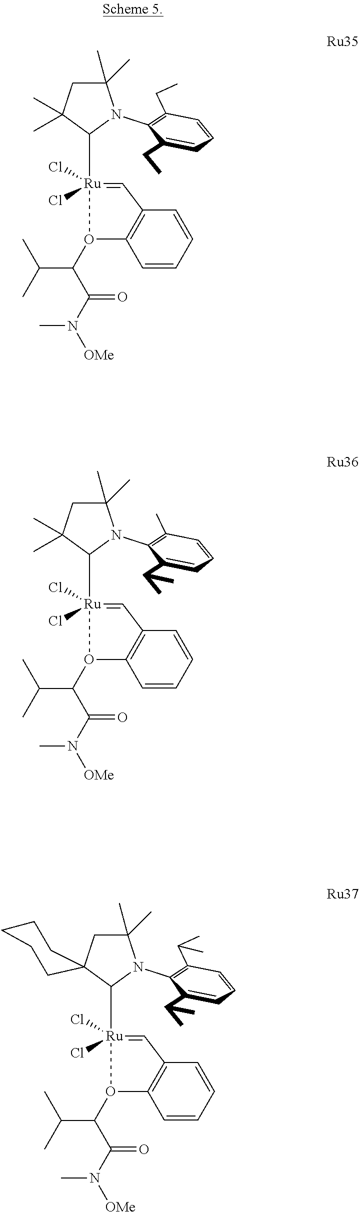

Subsequent modifications of ruthenium catalysts involved Hoveyda-Grubbs type complexes, wherein the hydrogen atom in the styrene part was replaced with an EWG or an EDG group. By modulating the nature of the styrenyl ether ligand, Mignagni et al. studied the reactivity of Ru38 and Ru39 catalysts [FR2947189A1; FR2934178A1]. It was shown that the presence of amino group donating electrons has a negative impact on the catalytic activity. On the other hand, the modification of Ru40 with an electron acceptor SO₂NMe₂ group (Zhan type catalyst) enabled the synthesis of an extremely active catalyst in the ethenolysis reaction of fatty acids [EP1905777B1; US2011/0306815A1].

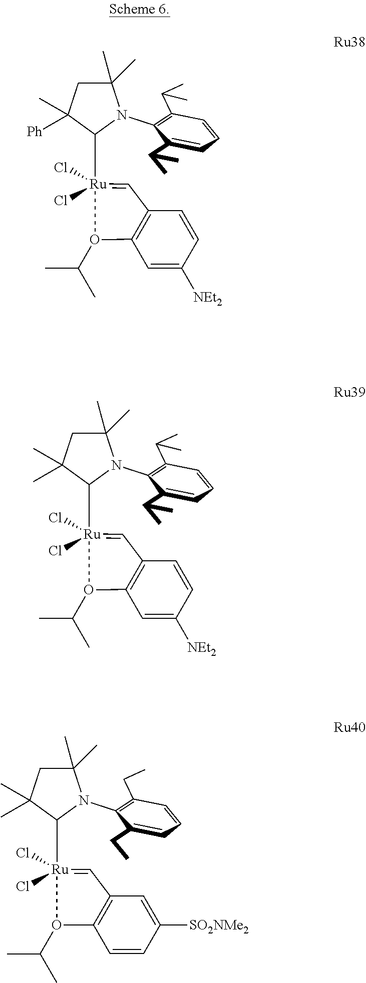

Verpoort et al. studied the impact of labile chelating groups: benzyl ether, benzyl thioether, and benzylamine [WO2017/185324A1]. All catalysis transformed methyl oleate with high selectivity and high TON values [Turnover number—the number of catalytic cycles, the number of moles of substrate undergoing reactions, calculated per one mole of the catalyst](from 180,000 to 210,000). A reaction performed with ethylene (99.995%), in the presence of Ru43 chelated with benzylamine and an activator (HSiCl₃), yielded the highest recorded thus far TON value (390,000).

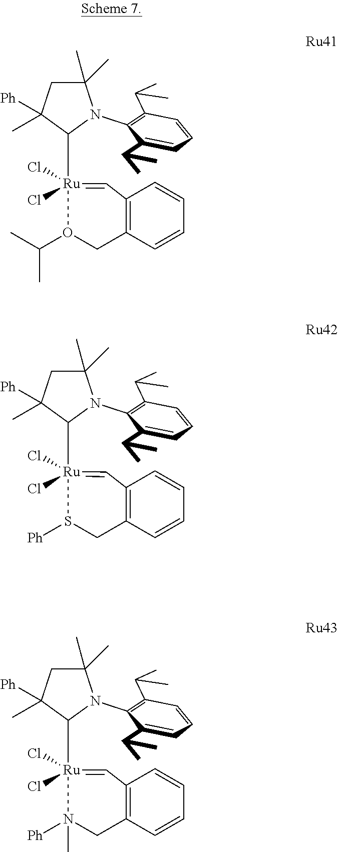

Lemcoff et al. showed that analogues of Hoveyda-Grubbs type catalysts chelated with sulfur in the benzylidene part are present in the cis/trans pairs Ru44-Ru47. The activity of complexes was studied i.e. in polymerization reactions of norbornene derivatives [Rozenberg, I. et al. ACS Catal. 2018, 8, 8182-8191].

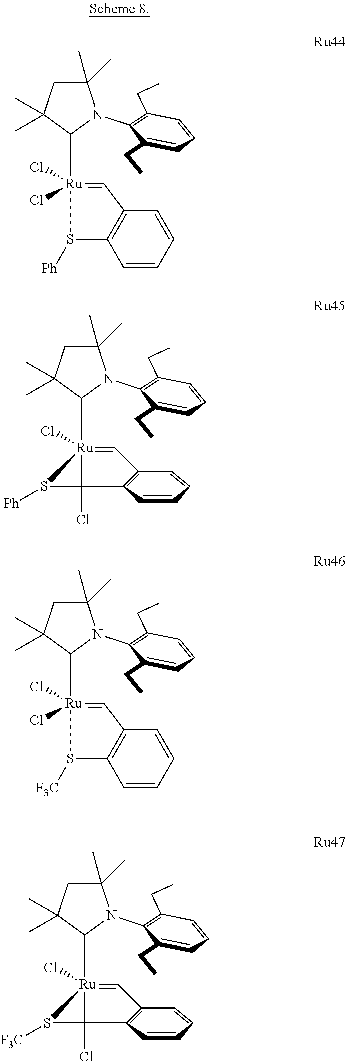

The limited availability of a wide range of structurally various aldehydes used as substrates in the synthesis of CAAC ligands is a significant problem known in state-of-the-art. Their use is mostly limited to simple aldehydes comprising isobutanal or 2-phenylpropanal. The high prices and low availability of other aldehyde derivatives, especially those with heterocyclic rings, as well as long and complicated synthesis pathways for these and other derivatives, in which the expected products are obtained with low yields, significantly limit the possibility of designing novel ruthenium catalysts containing modified CAAC ligands. These characteristics pose a significant limitation to the further development of organometallic catalysis based on ruthenium with novel CAAC ligands. Obtaining novel catalysts for olefin metathesis with ligands based, in particular, on various types of aldehydes with planned properties is becoming difficult to implement in chemical synthesis and economically unjustifiable in industrial scale-up.

In the search for novel ruthenium complexes with high catalytic activity and improved stability and selectivity enabling high TON values to be obtained, it is important that convenient synthetic paths based on easily available and inexpensive substrates lead to these compounds. From the point of view of industrial scaling, it is also important for the planned synthesis to be efficient at every stage and that the reaction products can be purified in a simple way, using techniques such as crystallization or distillation. It is also important to expand the library of ligands, the use of which will comprise an alternative and/or improved source of structures of ruthenium complexes used as catalysts in i.e. ethenolysis reactions of ester derivatives of fatty acids.

From an industrial point of view, it is extremely important that the activity of the new ruthenium complexes allows ethenolysis processes to be carried out with unpurified vegetable oils through a multi-step, laborious procedure of purifying raw materials from compounds that deactivate active catalyst molecules, such as oxygen, water, amines, sulfur compounds, halogenated compounds, peroxides, and others. It is equally important from an industrial point of view that the metathesis process can be carried out with ethylene available in an industrial installation, and not just commercially available high-purity ethylene (ethylene 3.0=99.9% purity or ethylene 4.5=99.995% purity). It is worth emphasizing that it is not possible to reproduce and apply under industrial conditions the procedures carried out in a glovebox under argon atmosphere, which are described in reputable journals by numerous respected scientists. Therefore, it is essential to search for novel ruthenium catalysts that, on the one hand, will be robust under reaction conditions, and at the same time active and efficient for performing the metathesis process.

Equally important is that transport and storage of catalysts should be easy and possible under normal atmospheric conditions, without the use of inert gases and low temperatures. Such properties of catalysts expected by the industry will enable their easy use on a large industrial scale, which will not require complex apparatus.

It was surprisingly discovered that complexes containing CAAC ligands with a heteroaromatic substituent were found to exhibit the properties desired and sought after by industry. Unexpectedly, it turned out that the ethenolysis processes catalyzed by the novel ruthenium complexes tolerate the presence of oxygen, so that the apparatus can be assembled in air, moreover, ethylene of lower purity than in the processes known in the literature can be used, as well as derivatives of fatty acids esters (oils of plant origin) without the need for a long purification process to high purity of raw materials.

### SUMMARY OF THE INVENTION

The subject of this invention is a precursor of cyclic alkyl amine carbenes (CAAC) with the formula CAAC-1

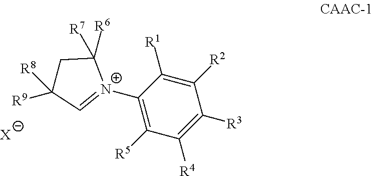

in which
X denotes an anion selected from a group comprising a halogen anion, BF₄⁻, PF₆⁻, ClO₄⁻, CF₃SO₂O⁻; R¹, R², R³, R⁴ and R⁵ denote independently a hydrogen atom, a C₁-C₁₂ alkyl group, a C₃-C₁₂ cycloalkyl group, a C₅-C₂₀ aryl group or a C₅-C₂₀ heteroaryl group, a C₅-C₂₅ aralkyl group, which may be independently substituted by one and/or more substituents selected from a group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulfide group (—SR″), an amino group (—NR″₂), in which the R″ group independently denotes a hydrogen atom, C₁-C₅ alkyl, C₆-C₂₄ aryl, C₇-C₂₄ aralkyl,
alternatively R¹, R², R³, R⁴ and R⁵ are connected and form a C₅-C₂₅ ring;
wherein each substituent R⁶, R⁷, and R⁸ denotes a hydrogen atom, a halogen atom, a C₁-C₁₂ alkyl group or a C₅-C₂₀ aryl group, which may be independently substituted by one and/or more substituents selected from a group comprising a hydrogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulfide group (—SR″), an amino group (—NR″₂), in which the R″ group independently denotes a hydrogen atom, C₁-C₅ alkyl, C₆-C₂₄ aryl, C₇-C₂₄ aralkyl;
substituent R⁹ denotes a substituted or unsubstituted heterocyclic group or an organometallic complex group such as ferrocene, which may be independently substituted by one and/or more substituents selected from a group comprising a hydrogen atom, a hydrogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulfide group (—SR″), an amino group (—NR″₂), in which the R″ group independently denotes a hydrogen atom, C₁-C₅ alkyl, C₆-C₂₄ aryl, C₇-C₂₄ aralkyl,
alternatively, R⁶ and R⁷ and/or R⁸ and R⁹ are connected and form a C₅-C₂₅ ring.

Preferably the substituent R⁹ denotes a substituted or unsubstituted heterocyclic group selected from a heterocyclic group selected from thiophene, benzothiophene, furan, benzofuran, pyrrole, benzopyrrole, aziridine, oxirane, thiirane, azetidine, oxetane, thietane, pyrrolidine, tetrahydrofuran, tetrahydrothiophene, piperidine, tetrahydropyran, thiane, pyridine, azepane, oxepane, thiepane, azepine, oxepin, thiepine, oxazole, imidazole, thiazole, isoxazole, pyrazole, isothiazole, triazyne, pyrrolidine, pyridine, pyrimidine, hydantoin, quinoline, isoquinoline, chromonyl, coumarin, indole, indolysin, indazole, purine, quinolysine, isoquinol, quinol, phthalazine, naphthyridine, carbazole, β-carboline, or substitute R⁹ denotes a substituted or unsubstituted organometallic complex group comprising cyclopentyl rings and metal atom selected from iron, cobalt, nickel, chromium, titanium, zirconium (metallocenes).

Preferably the substituent R⁹ denotes a substituted or unsubstituted heterocyclic group selected from thiophene, benzothiophene, furan, benzofuran or ferrocene.

Preferably, the precursor defined above has a structure represented by the formula L1, L2, L3, L4, L5, L6, L7, L8, L9, L10, L11, L12, L13, L14, L15, L16, L17, L18, L19, L20, L21 or L22:

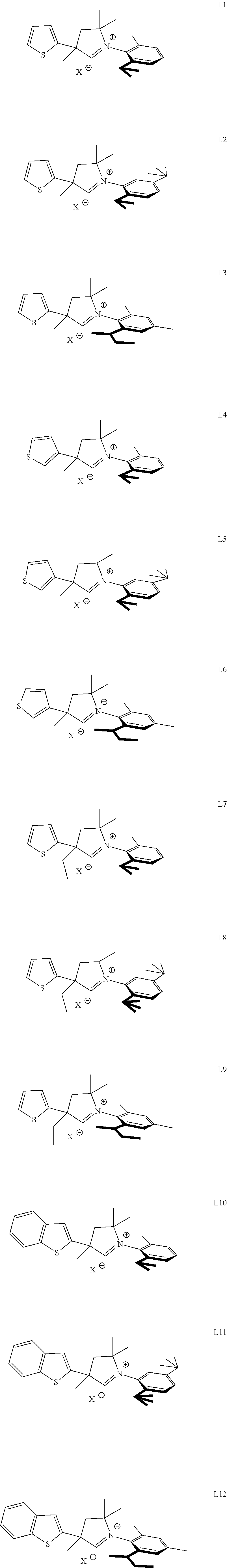

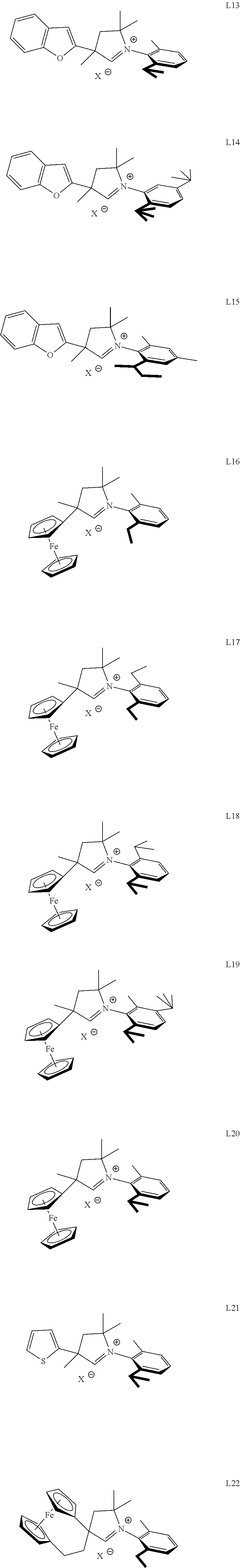

The subject of the invention is also a ruthenium complex of formula 1-Ru

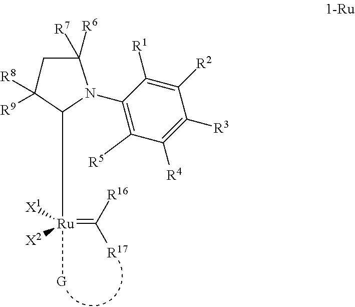

in which:
X¹ and X² independently from each other denote an anionic ligand selected from a group comprising a halogen anion, a group —CN, —SCN, —OR^{a}, —SR^{a}, —O(C═O)R^{a}, —O(SO₂)R^{a} and —OSi(R^{a})₃, in which R^{a} denotes C₁-C₁₂ alkyl, C₃-C₁₂ cycloalkyl, C₂-C₁₂ alkenyl or C₅-C₂₀ aryl, which is optionally substituted by at least one C₁-C₁₂ alkyl, C₁-C₁₂ perfluoroalkyl, C₁-C₁₂ alkoxyl, C₅-C₂₄ aryloxyl, C₅-C₂₀ heteroaryloxyl or a halogen atom;
R¹, R², R³, R⁴ and R⁵ denote independently a hydrogen atom, a C₁-C₁₂ alkyl group, a C₃-C₁₂ cycloalkyl group, a C₅-C₂₀ aryl group or a C₅-C₂₀ heteroaryl group, a C₅-C₂₅ aralkyl group, which may be independently substituted by one and/or more substituents selected from a group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulfide group (—SR″), an amino group (—NR″₂), in which the R″ group independently denotes a hydrogen atom, C₁-C₅ alkyl, C₆-C₂₄ aryl, C₇-C₂₄ aralkyl, alternatively R¹, R², R³, R⁴ and R⁵ are connected and form a C₅-C₂₅ ring;
each substituent R⁶, R⁷, and R⁸ denotes a hydrogen atom, a halogen atom, an alkyl C₁-C₁₂ group or a C₅-C₂₀ aryl group, which may be substituted independently by one and/or more substituents selected from a group comprising a hydrogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulfide group (—SR″), an amino group (—NR″₂), in which the R″ group independently means a hydrogen atom, C₁-C₅ alkyl, C₆-C₂₄ aryl, C₇-C₂₄ aralkyl;
substituent R⁹ denotes a substituted or unsubstituted heterocyclic group and/or organometallic complex group which may be independently substituted by one and/or more substituents selected from a group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulfide group (SR″), an amino group (—NR″₂), in which the R″ group independently denotes a hydrogen atom, C₁-C₅ alkyl, C₆-C₂₄ aryl, C₇-C₂₄ aralkyl,
alternatively R⁶ and R⁷ and/or R⁸ and R⁹ are connected and form a C₅-C₂₅ ring;
R¹⁶ and R¹⁷ denote independently a hydrogen atom, a halogen atom, C₁-C₂₅ alkyl, C₃-C₁₂ cycloalkyl, C₁-C₁₂ perfluoroalkyl, C₂-C₂₅ alkene, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₅ aryl, C₅-C₂₅ aryloxyl, C₆-C₂₅ arylalkyl, C₅-C₂₅ heteroaryl, C₅-C₂₅ heteroaryloxyl, C₅-C₂₅ perfluoroaryl, a 3-12-membered heterocycle comprising a sulfur, oxygen, nitrogen, selenium or a phosphorus atom;
wherein the substituents R¹⁶ and R¹⁷ may be connected forming a ring selected from a group comprising C₃-C₂₅ cycloalkyl, C₃-C₂₅ cycloalkenyl, C₃-C₂₅ cycloalkynyl, C₅-C₂₅ aryl, C₅-C₂₅ heteroaryl, C₅-C₂₅ perfluoroaryl, a 3-12-membered heterocycle containing a sulfur, oxygen, nitrogen, selenium or phosphorus atom, which may be independently substituted by one or more substituents selected from a group comprising a hydrogen atom, a halogen atom, C₁-C₂₅ alkyl, C₃-C₂₅ cycloalkyl, C₁-C₁₂ perfluoroalkyl, C₂-C₂₅ alkene, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₅ aryl, C₅-C₂₅ aryloxyl, C₆-C₂₅ arylalkyl, C₅-C₂₅ heteroaryl, C₅-C₂₅ heteroaryloxyl, C₅-C₂₅ perfluoroaryl, a 3-12-membered heterocycle;
G is selected from such as
   a ligand with the formula CAAC-1

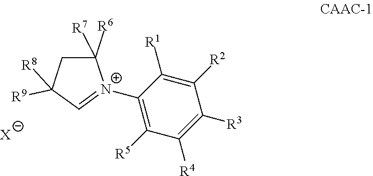

in which X and the substituents R¹ to R⁹ have the meanings defined above
or
   heteroatom 1
selected from a group comprising an oxygen, sulfur or selenium atom, substituted by a group selected from such as a hydrogen atom, a halogen atom, an oxygen atom, C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or heteroaryloxyl C₅-C₂₄, a 3-12-membered heterocycle, optionally substituted by an acyl group (—COR′), a cyano group (—CN), a carboxyl group (—COOH), an ester group (—COOR′), an ester group (—CH₂COOR′), an ester group (—CHR′COOR′), an ester group (—C(R′)₂COOR′), an amide group (—CONR′₂), a Weinreb-type amide (—CON(R′)(OR′)), a sulfone group (—SO₂R′), a formyl group (—CHO), a sulfonamide group (—SO₂NR′₂), a ketone group (—COR′), a thioamide group (—CSNR′₂), a thioketone (—CSR′), a thionoester group (—CSOR′), a thioester group (—COSR′), a dithioester group (—CS₂R′), in which the group R′ denotes independently C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl, C₅-C₂₄ heteroaryloxyl and then the dashed line denotes a direct bond between a heteroatom and the substituent R¹⁷ or it denotes a bond between the substituent R¹⁷ and a heteroatom via a methylene bridge —CH₂—, —CHR′—, or —CR′₂— wherein the substituent R¹⁴ is C₅-C₁₅ aryl, optionally substituted by 1-4 substituents independently selected from a group comprising a hydrogen atom, a halogen atom, C₁-C₂₅ alkyl, C₃-C₂₅ cycloalkyl, C₂-C₂₅ alkenyl, C₃-C₂₅ cycloalkenyl, C₂-C₂₅ alkynyl, C₃-C₂₅ cycloalkynyl, C₁-C₂₅ perfluoroalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₆-C₂₀ heteroaryl or C₅-C₂₄ heteroaryloxyl, a 3-12-membered heterocycle, an alkoxyl group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes C₁-C₅ alkyl, C₁-C₅ perfluoroalkyl, C₆-C₂₄ aryl, C₇-C₂₄ aralkyl, C₅-C₂₄ perfluoroaryl, or
   heteroatom 2
selected from a group comprising a nitrogen or a phosphorus atom, substituted by the group selected from such as a hydrogen atom, methylidene, optionally substituted by the substitute R′, C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, a 3-12-membered heterocycle, an acyl group (—COR′), an ester group (—COOR′), a tert-butyloxycarbonyl group (t-Boc) or a 9-fluorenylmethoxycarbonyla group (Fmoc), a carbamine group (—CONR′₂), a sulfone group (—SO₂R′), a formyl group (—CHO), in which the R′ group denotes C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, optionally substituted with an acyl group (—COR′), a cyano group (—CN), a carboxyl group (—COOH), an ester group (—COOR′), an ester group (—CH₂COOR′), an ester group (—CHR′COOR′), an ester group (—C(R′)₂COOR′), an amide group (—CONR′₂), a sulfone group (—SO₂R′), a formyl group (—CHO), a sulfonamide group (—SO₂NR′₂), a ketone group (—COR′), a thioamide group (—CSNR′₂), a thioketone group (—CSR′), a thionoester group (—CSOR′), a thioester group (—COSR′) or a dithioester group (—CS₂R′), in which the R′ group denotes C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, and then the dashed line denotes a direct bond between the heteroatom and the substituent R¹⁴ or it denotes a bond between the substituent R¹⁷ with a heteroatom via methylene bridge (CH₂)—, —(CHR′)—, or —(CR′₂)—; wherein the substituent R¹⁷ is C₅-C₁₅ aryl, optionally substituted with 1-4 substituents independently selected from the group comprising a hydrogen atom, a halogen atom, C₁-C₂₅ alkyl, C₃-C₂₅ cycloalkyl, C₂-C₂₅ alkenyl, C₃-C₂₅ cycloalkenyl, C₂-C₂₅ alkynyl, C₃-C₂₅ cycloalkynyl, C₁-C₂₅ perfluoroalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₆-C₂₀ heteroaryl or C₅-C₂₄ heteroaryloxyl, a 3-12-membered heterocycle, an alkoxyl group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″) or a dithioester group (—CS₂R″), in which the group R″ denotes C₁-C₅ alkyl, C₁-C₅ perfluoroalkyl, C₆-C₂₄ aryl, C₇-C₂₄ aralkyl, C₅-C₂₄ perfluoroaryl, or
   heteroatom 3
selected from a group comprising a halogen atom and then the dashed line denotes a direct bond between the heteroatom and the R¹⁷ substituent, wherein the R¹⁷ substituent is C₅-C₁₅ aryl, or C₅-C₂₅ polyaryl, optionally substituted with 1-4 substituents selected independently from a group comprising a hydrogen atom, a halogen atom, C₁-C₂₅ alkyl, C₃-C₂₅ cycloalkyl, C₂-C₂₅ alkenyl, C₃-C₂₅ cycloalkenyl, C₂-C₂₅ alkynyl, C₃-C₂₅ cycloalkynyl, C₁-C₂₅ perfluoroalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₆-C₂₀ heteroaryl or C₅-C₂₄ heteroaryloxyl, a 3-12-membered heterocycle, an alkoxy group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes C₁-C₅ alkyl, C₁-C₈ perfluoroalkyl, C₆-C₂₄ aryl, C₇-C₂₄ aralkyl, C₅-C₂₄ perfluoroaryl.

The ruthenium complex is preferably represented by the formula 1a-Ru

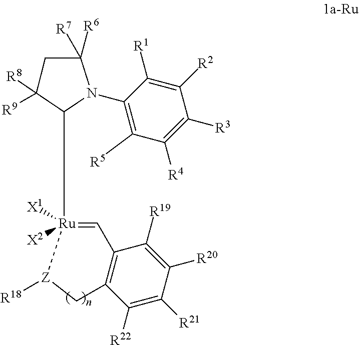

in which X¹ and X² and the R¹, R², R³, R⁴, R⁵, R⁶, R⁷, R⁸, R⁹ substituents have the meanings defined above
‘n’ means 1 or 0
Z is selected from a group comprising halogen atoms, O atom, S atom, Se atom, or a NR′″ group, in which R′″ denotes methylidene, C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, a 3-12-membered heterocycle, an acyl group (—COR′), an ester group (—COOR′), a tert-butylcarboxycarbon group (t-Boc) or a 9-fluorenylmethoxycarbonyl group (Fmoc), a carbamine group (—CONR′₂), a sulfone group (—SO₂R′), a formyl group (—CHO), in which the R′ group denotes C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl,
or a halogen atom, wherein if Z denotes a halogen atom, R¹⁸ is absent;
R¹⁸ means independently a hydrogen atom, C₁-C₂₅ alkyl, C₁-C₂₅ cycloalkyl, C₅-C₂₀ alkoxyl, C₅-C₂₀ aryl, C₅-C₂₄ aryloxyl, a —COOR′″ group, a —CH₂COOR′″ group, a —CONR′″₂ group, a —CH₂CONR′″₂ group, a —COR′″ group, a —CH₂COR′″ group, a —CON(OR′″)(R′″) group, a —CH₂CON(OR′″)(R′″) group or a halogen atom, wherein R′″ denotes C₁-C₁₂ alkyl, C₃-C₁₂ cycloalkyl, C₂-C₁₂ alkenyl, C₆-C₂₀ aryl, which are optionally substituted with at least one C₁-C₁₂ alkyl, C₁-C₁₂ perfluoroalkyl, C₁-C₁₂ alkoxyl, C₆-C₂₄ aryloxyl, or a halogen atom;
R¹⁹, R²⁰, R²¹, and R²² denote independently a hydrogen atom, a halogen atom, a C₁-C₂₅ alkyl group, a C₂-C₂₅ alkenyl group, a C₅-C₂₅ aryl group, an alkoxy group (—OR″), a sulfide group (—SR″), a sulfoxide (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R′), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes C₁-C₅ alkyl, C₁-C₅ perfluoroalkyl, C₆-C₂₄ aryl, C₇-C₂₄ aralkyl, C₅-C₂₄ perfluoroaryl, wherein the R¹⁶, R¹⁷, R¹⁸ and R¹⁹ substituents may be connected, thus forming a substituted or unsubstituted C₄-C₁₀ cyclic or C₄-C₁₂ polycyclic system.

The ruthenium complex is preferably represented by the formula 1b-Ru

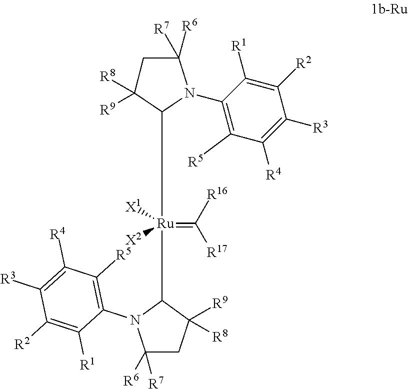

in which X¹ and X² and the R¹, R², R³, R⁴, R⁵, R⁶, R⁷, R⁸, R⁹ substituents have the meanings defined above
R¹⁶ and R¹⁷ independently denote a hydrogen atom, a halogen atom, C₁-C₂₅ alkyl, optionally substituted; C₃-C₂₅ cycloalkyl, optionally substituted; C₁-C₁₂ perfluoroalkyl, optionally substituted; C₂-C₂₅ alkene, optionally substituted; C₂-C₂₅ alkenyl, optionally substituted; C₃-C₂₅ cycloalkenyl, optionally substituted; C₂-C₂₅ alkynyl, optionally substituted; C₃-C₂₅ cycloalkynyl, optionally substituted; C₁-C₂₅ alkoxyl, optionally substituted; C₅-C₂₅ aryl, optionally substituted; C₅-C₂₅ aryloxyl, optionally substituted; C₆-C₂₅ arylalkyl, optionally substituted; C₅-C₂₅ heteroaryl, optionally substituted; C₅-C₂₅ heteroaryloxyl, optionally substituted; C₅-C₂₅ perfluoroaryl, optionally substituted; a 3-12-membered heterocycle containing a sulfur, oxygen, nitrogen, selenium or a phosphorus atom, optionally substituted;
wherein the R¹⁶ and R¹⁷ substituents may be connected, forming a ring selected from a group comprising C₃-C₂₅ cycloalkyl, cycloalkenyl C₃-C₂₅, C₃-C₂₅ cycloalkynyl, C₅-C₂₅ aryl, C₅-C₂₅ heteroaryl, C₅-C₂₅ perfluoroaryl, a 3-12-membered heterocycle comprising a sulfur, oxygen, nitrogen, selenium or a phosphorus atom, which may be independently substituted with one or more substituents selected from a group comprising a hydrogen atom, a halogen atom, C₁-C₂₅ alkyl, C₃-C₂₅ cycloalkyl, C₁-C₁₂ perfluoroalkyl, C₂-C₂₅ alkene, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₅ aryl, C₅-C₂₅ aryloxyl, C₆-C₂₅ arylalkyl, C₅-C₂₅ heteroaryl, C₅-C₂₅ heteroaryloxyl, C₅-C₂₅ perfluoroaryl, a 3-12-membered heterocycle.

The ruthenium complex is preferably represented with the formulas Ru1a, Ru2a, Ru3a, Ru4a, Ru5a, Ru6a, Ru7a, Ru8a, Ru9a, Ru10a, Ru11a, Ru11a, Ru12a, Ru13a, Ru14a, Ru15a, Ru16a, Ru17a, Ru18a, Ru19a, Ru20a, Ru21a, Ru22a:

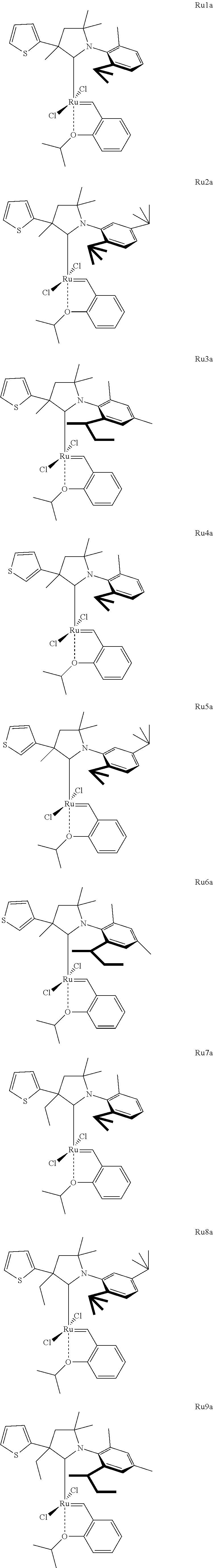

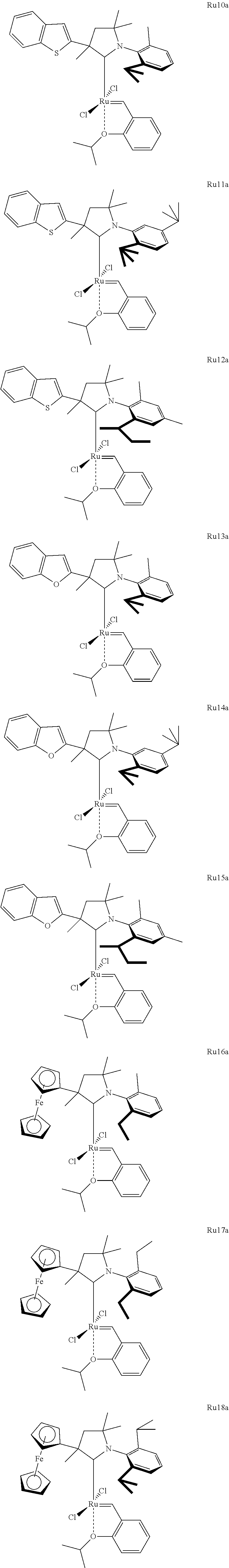

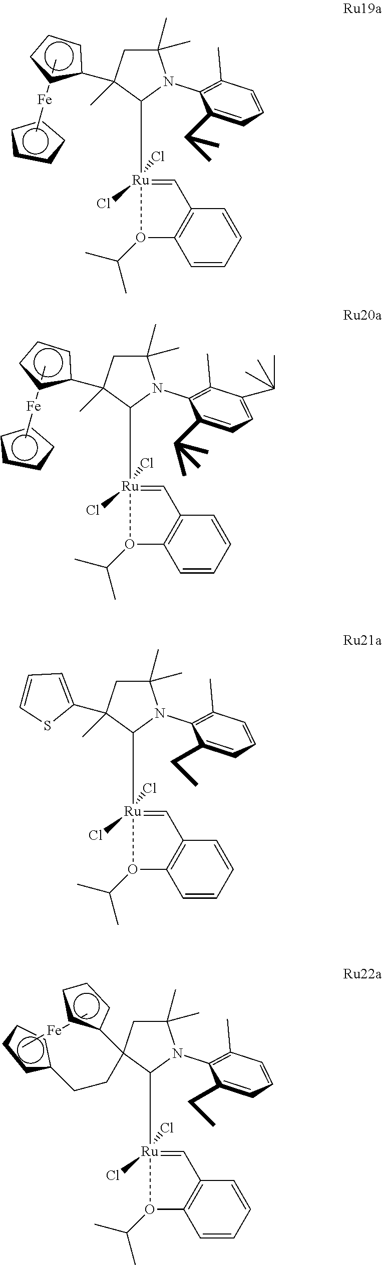

The subject of the invention is also a method of obtaining a ruthenium complex of formula 1a-Ru

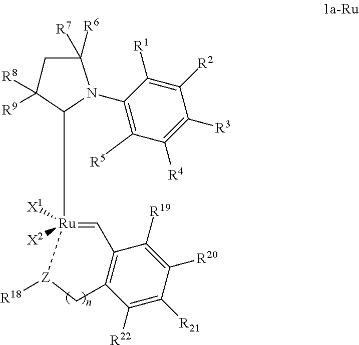

defined above, in which the alkylidene ruthenium complex with the formula 10

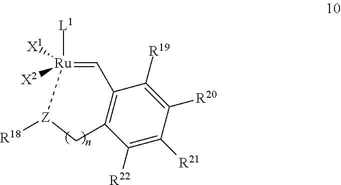

in which:
L¹ denotes a neutral ligand selected from a group comprising pyridine or substituted pyridine, P(R′)₃, P(OR′)₃, O(R′)₂, N(R′)₃, wherein each R′ independently denotes C₁-C₁₂ alkyl, C₃-C₁₂ cycloalkyl, C₅-C₂₀ aryl, C₇-C₂₄ aralkyl, C₅-C₂₄ perfluoroaryl, a 5-12-membered heteroaryl;
N, Z, X¹, X² and the substituents R¹⁸, R¹⁹, R²⁰, R²¹ and R²² have the meanings defined above undergoes reaction with carbene of formula 8

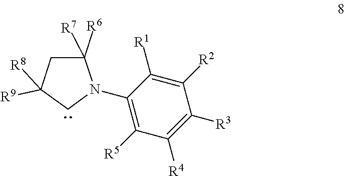

in which the substituents R¹ to R⁹ have the meanings defined above.

The subject of the invention also relates to the use of the compound of formula 1-Ru, defined above, as a precatalyst and/or a catalyst in olefin metathesis reactions, in particular in ring-closing metathesis (RCM), cross-metathesis (CM), homometathesis (cross-metathesis between two molecules of the same olefin), ethenolysis, isomerization, in the reaction of diastereoselective ring-rearrangement metathesis (DRRM) reactions, ‘alkene-alkyne’ (en-yn) metathesis or ROMP or ADMET polymerization reactions.

The reaction is preferably carried out in an organic solvent, such as toluene, mesitylene, hexane, cyclohexane, ethyl acetate, methyl acetate, methyl carbonate, ethyl carbonate, tert-butyl-methyl ether, cyclopentyl-methyl ether, diethyl ether, THF, 2-Me-THF, 4-Me-THP, dioxane, DME, PAO. PEG, paraffin, esters of saturated fatty acids.

The reaction is preferably carried out in a solvent-free system.

The reaction is preferably carried out at a temperature between 20° C. and 200° C.

The reaction is preferably carried out over a period between 5 minutes and 48 hours.

The 1-Ru compound is preferably used in a quantity not greater than 10 mol %.

The 1-Ru compound is preferably used in a quantity not greater than 0.1 mol %.

The 1-Ru compound is preferably added to the reaction mixture in aliquots, as a solid and/or continuously, using a pump, as a solution in an organic solvent.

The gaseous by-product of the reaction, selected from ethylene, propylene, butylene, is preferably actively removed from the reaction mixture using inert gas barbotage or under reduced pressure.

The subject of the invention is explained in embodiments in the Figures, in which:

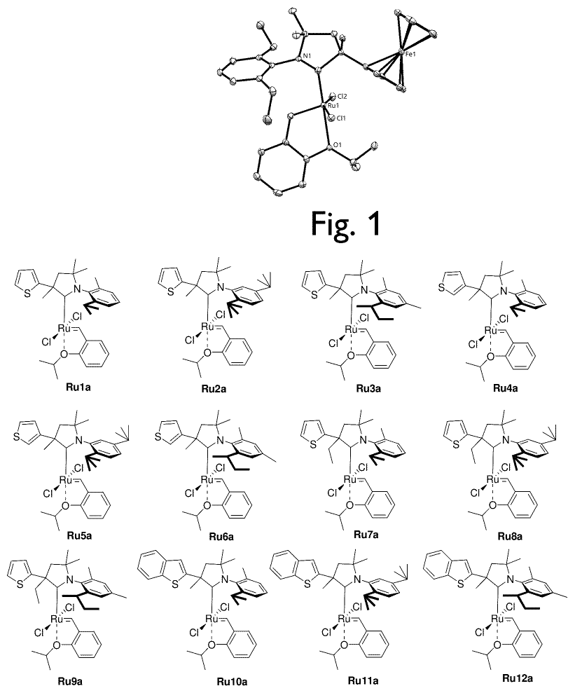

FIG. 1 presents the structure of the Ru17a compound obtained on the basis of X-ray structural analysis;

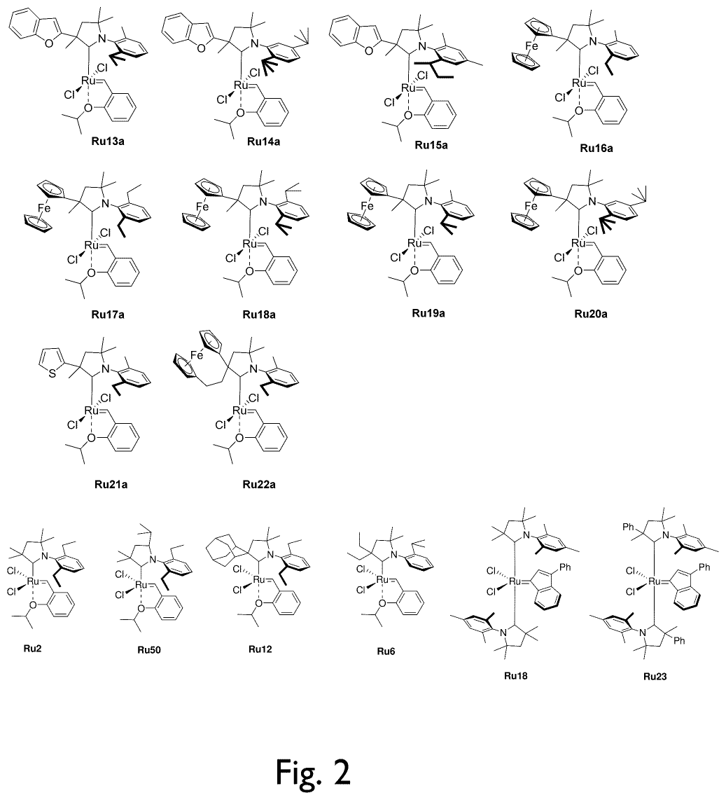

FIG. 2 presents a summary of precatalysts and catalysts of olefin metathesis available on the market, and of novel precatalysts and catalysts according to the invention;

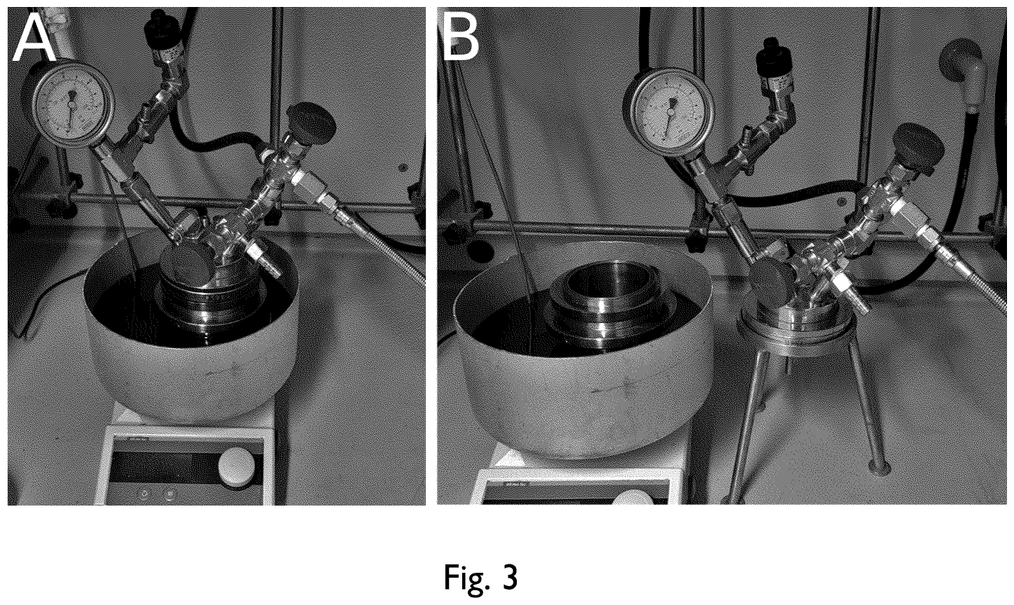

FIG. 3 presents a photography showing: (A) the set up and closed reaction system—autoclave, and (B) the reaction system (autoclave), which is open prior to the ethenolysis reaction and does not require a glovebox at any stage of the reaction process.

In this description, terms used have the following meanings.

Terms undefined in this document have meanings which are provided and understood by the specialist in the field in light of the best knowledge available, this disclosure and the context of the patent application description.

Unless otherwise stated, the following conventions of chemical terms are used in this description and have the meanings indicated as in the definitions below:

As used in this description, the term “halogen atom” means an element selected from F, Cl, Br, I.

The term “carbene” means an electrically inert molecule in which the carbon atom has two non-bonding electrons occurring in the singlet or triplet state and is linked by a single covalent bond to two groups or linked by a double covalent bond to one group. The term ‘carbene’ also includes carbene analogues in which the carbene carbon atom is replaced by another chemical element such as boron, silicon, germanium, tin, lead, nitrogen, phosphorus, sulfur, selenium or tellurium.

The term “alkyl” refers to a saturated, linear or branched hydrocarbon substituent with the indicated number of carbon atoms. Examples of alkyl substituents include -methyl, -ethyl, -n-propyl, -n-butyl, -n-pentyl, -n-hexyl, -n-heptyl, -n-octyl, -n-nonyl and -n-decyl. Representative branched —(C₁-C₁₀) alkyls include -isopropyl, -sec-butyl, -isobutyl, -tertbutyl, -isopentyl, -neopentyl, -1-methylbutyl, -2-methylbutyl, -3-methylbutyl, -1,1-dimethylpropyl, -1,2-dimethylpropyl, -1-methylpentyl, -2-methylpentyl, -3-methylpentyl, -4-methylpentyl, -1-ethylbutyl, -2-ethylbutyl, -1,1-dimethylbutyl, -1,2-dimethylbutyl, -1,3-dimethylbutyl, -2,2-dimethylbutyl, -2,3-dimethylbutyl, -3,3-dimethylbutyl, -1-methylhexyl, -2-methylhexyl, -3-methylhexyl, -4-methylhexyl, -1,2-dimethylpentyl, -1,3-dimethylpentyl, -5-methylhexyl, -1,2-dimethylhexyl, -1,3-dimethylhexyl, -3,3-dimethylhexyl, -1,2-dimethylheptyl, -1,3-dimethylheptyl, -3,3-dimethylheptyl and the like.

The term “alkoxyl” refers to an alkyl substituent as defined above, connected via an oxygen atom.

The term “perfluoroalkyl” denotes an alkyl group as defined above, in which all the hydrogen atoms have been replaced with the same or different halogen atoms.

The term “cycloalkyl” refers to a saturated, mono- or polycyclic hydrocarbon substituent with the indicated number of carbon atoms. Examples of cycloalkyl substituent include -cyclopropyl, -cyclobutyl, -cyclopentyl, -cyclohexyl, -cycloheptyl, -cyclooctyl, -cyclononyl, -cyclodecyl and the like.

The term “alkenyl” refers to an unsaturated, linear or branched, acyclic hydrocarbon substituent with the indicated number of hydrogen atoms and containing at least one double carbon-carbon bond. Examples of alkenyl substituents include -vinyl, -allyl, -1-butenyl, -2-butenyl, -isobutenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “cycloalkenyl” refers to an unsaturated, cyclic or branched, cyclic hydrocarbon substituent with the indicated number of hydrogen atoms and containing at least one double carbon-carbon bond. Examples of cycloalkenyl substituents

include -cyclopropene, -cyclobutene, -cyclopentene, -cyclohexene, -cycloheptene, -cyclooctene, -cyclononene, -cyclodecene, -metylcyclopropene, -ethylcyclobutene, -isopropylcyclo pentene, -methylcyclohexene and the like.

The term “aryl” refers to an aromatic, mono- or polycyclic hydrocarbon substituent with the indicated number of carbon atoms. Examples of aryl substituents

include -phenyl, -tolyl, -xylyl, -naphthtyl, -2,4,6-trimethylphenyl, -2-fluorophenyl, -4-fluorophenyl, -2,4,6-trifluorophenyl, -2,6-difluorophenyl, -4-nitrophenyl and the like.

The term “aralkyl” refers to an alkyl substituent as defined above, substituted with at least one aryl as defined above. Examples of aralkyl substituents include -benzyl, -diphenylmethyl, -triphenylmethyl and the like.

The term “heteroaryl” refers to an aromatic mono- or polycyclic hydrocarbon substituent with the indicated number of carbon atoms, in which at least one carbon atom has been replaced with a heteroatom selected from O, N and S atoms. Examples of heteroaryl substituents include -furyl, -thienyl, -imidazolyl, -oxazolyl, -thiazolyl, -isoxazolyl, -triazolyl, -oxadiazolyl, -thiadiazolyl, -tetrazolyl, -pyridyl, -pyrimidyl, -triazinyl, -indolyl, -benzo[b]furyl, -benzo[b]thienyl, -indazolyl, -benzoimidazolyl, -azaindolyl, -quinolyl, -isoquinolyl, -carbazolyl and the like.

The term “heterocycle” refers to a saturated, unsaturated or partially unsaturated hydrocarbon substituent, with the indicated number of carbon atoms, in which at least one carbon atom has been replaced with a heteroatom selected from O, N and S atoms. Example heterocycle substituents include -furyl, -thiophenyl, -pyrrolyl, -oxazolyl, -imidazolyl, -thiazolyl, -isoxazolyl, -pyrazolyl, -isothiazolyl, -triazinyl, -pyrrolidinonyl, -pyrrolidinyl, -hydantoinyl, -oxiranyl, -oxetanyl, -tetrahydrofuranyl, -tetrahydrothiophenyl, -quinolinyl, -isoquinolinyl, -chromonyl, -coumarinyl, -indolyl, -indolysinyl, -benzo[b]furanyl, -benzo[b]thiophenyl, -indazolyl, -purinyl, -4H-quinolysinyl, -isoquinolyl, -quinolyl, -phtalazinyl, -naphtyridinyl, -carbazolyl, -β-carbolinyl and the like.

The term “neutral ligand” refers to an non-charged substituent capable of coordinating with a metallic center (transition metal atom). Examples of such ligands may include: N-heterocyclic carbenes (NHC), cyclic (alkyl) (amino) carbenes (CAAC), amines, phosphines and their oxides, alkyl and aryl phosphites and phosphates, arsines and their oxides, ethers, alkyl and aryl sulfides, coordinated unsaturated or aromatic hydrocarbons, alkyl and aryl halides, nitriles, isonitriles, sulfides, sulfoxides, sulfones, thioketones, thioamides, thioester, thionoesters and dithioesters.

The term “anionic ligand” refers to a substituent capable of coordinating with a charged metallic center (transition metal atom), which can partially or fully compensate the charge of the metallic center. Examples of such ligands may include fluoride, chloride, bromide, iodide, cyanide, cyanate and thiocyanate anions, carboxylic acid anions, alcohol anions, phenol anions, thiol and thiophenol anions, anions of hydrocarbons with delocalized charge (e.g. cyclopentadiene anion), anions of (organo)sulfuric acids and (organo)phosphoric acids and their esters (such as e.g. anions of alkylsulfonic and arylsulfonic acids, anions of alkylphosphoric and arylphosphoric acids, anions of alkyl and aryl esters of sulfuric acid, anions of alkyl and aryl esters of phosphoric acids, anions of alkyl and aryl esters of alkylphosphoric and arylphosphoric acids).

The term “heteroatom” means an atom selected from the group comprising oxygen sulfur, nitrogen, phosphorous, boron, silicon, arsenic, selenium, tellurium.

The term “PAO” stands for polyolefins, an abbreviation for Poly-Alpha-Olefins, in the case of the present invention, an abbreviation used for low molecular weight polyolefins used as high boiling point solvents. It also denotes a class of solvents and/or lubricants that are the product of the polymerization of ethylene derivatives, leading to the formation of branched, saturated hydrocarbons, used here as heat-resistant, non-polar, high boiling point solvents.

### EMBODIMENTS OF THE INVENTION

The following examples are included only to illustrate the invention and to clarify particular aspects of the invention, not to limit it, and should not be equated with the entire scope of the invention as defined in the appended claims. In the following examples, unless otherwise indicated, standard materials and methods used in the field were used or followed manufacturers' recommendations for specific reactants and methods.

When necessary, model compounds for the metathesis reaction were purified by fractional distillation and then stored under inert gas atmosphere over activated neutral aluminum oxide. Tetrahydrofuran was purified by sodium-potassium alloy distillation in the presence of benzophenone, and then stored over 4 Å molecular sieves. When expedient, selected reactions were carried out under an argon atmosphere using reaction vessels heated at 130° C. Aluminum oxide (Al₂O₃, neutral, Brockman Grade I) was activated by heating at 150° C. under reduced pressure for 16 hours.

Starting compounds for the synthesis of aldehyde derivatives were commercially available.

### Example I

### Synthesis of New Aldehydes, Precursors of CAAC Ligands

The following Scheme 9 illustrates the first three steps in the synthesis of CAAC ligand precursors to synthesize ruthenium catalysts for olefin metathesis (with the general formulae Ru1a to Ru21a, Scheme 9), which are the subject of the present invention.

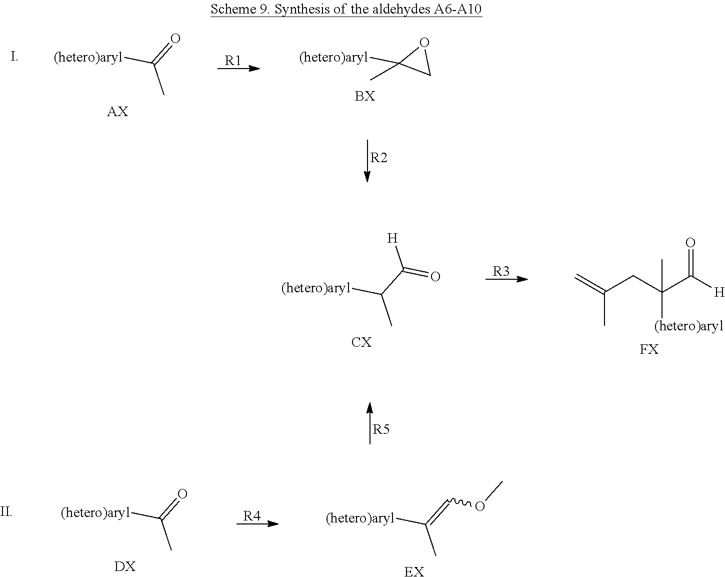

Reactions R1 through R5, shown in Scheme 9, were carried out using commercially available substrates based on procedures described in the literature with modifications developed by the authors of the invention. Unless otherwise written, the reactions described used commercially available solvents and paid no attention to the presence of oxygen and/or moisture.

### Reaction R1

In the R1 step (Scheme 9), the synthesis of an epoxide with the general formula BX is carried out. For this purpose, a ketone of the general formula AX and a trimethyl sulfonyl salt, preferably either trimethyl sulfonyl bromide (Me₃SBr) or iodide (Me₃SI), are used. The transformation is carried out in an organic solvent, preferably acetonitrile (MeCN) or another organic solvent, using a stoichiometric amount of the sulfonium salt and using a metal hydroxide, preferably potassium hydroxide (KOH). The reaction mixture is carried out at an elevated temperature, preferably 60° C. for 2 to 16 hours. The product is separated by filtration from the reaction mixture and distillation of the solvent.

### Embodiment of R1

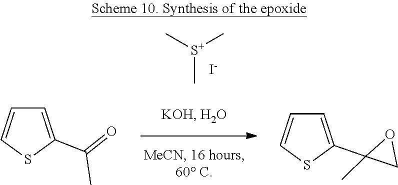

Into a reaction vessel equipped with a stirring element, 2-acetylthiophene (7.01 g, 6.00 mL, 55.0 mmol, 1.0 equiv.), Me₃SI (14.9 g, 71.5 mmol, 1.3 equiv.), KOH (9.44 g, 0.14 mmol, 2.6 equiv.), distilled water (0.25 mL) and MeCN (55 mL) were placed under an argon atmosphere. The contents of the vessel were stirred for 16 hours at 60° C. After the reaction was completed, 15 mL of diethyl ether (Et₂O) was added, the precipitate was filtered off and washed again with diethyl ether (40 mL), the solvent was evaporated under reduced pressure. The remaining dark pink oil was washed with diethyl ether (40 mL), the solvent was evaporated and washed with n-hexane (40 mL), the solvent was evaporated under reduced pressure to give the expected product as a yellow oil in 91% yield (6.98 g, 49.8 mmol).

¹H NMR (400 MHz, CDCl₃) δ ppm: 7.21 (dd, J=5.1, 1.2 Hz, 1H), 7.04 (dd, J=3.7, 1.3 Hz, 1H), 6.96 (dd, J=5.1, 3.6 Hz, 1H), 3.07-3.03 (m, 2H), 1.78-1.78 (m, 3H);

¹³C NMR (101 MHz, CDCl₃) δ ppm: 145.9, 127.2, 125.0, 124.8, 58.7, 55.2, 22.2;

### Reaction R2

In step R2 (Scheme 9), the synthesis of an aldehyde of the general formula CX is carried out. For this purpose, an epoxide of the general formula BX and Lewis acid, preferably SiO₂, chloroform, ZnCl₂ or ZnBr₂ are used. The transformation is carried out in an organic solvent, preferably ethyl acetate (EtOAc), chloroform or toluene (PhMe). The reaction mixture is carried out at room temperature until full conversion of the substrate (2.5-48 hours). The product is purified by distillation of the solvent and then used in the next step without further purification.

### Embodiment of Performing Reaction R2

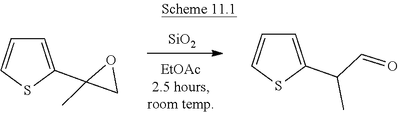

2-Methyl-2-(thiophen-2-yl)epoxide (6.6 g, 47.0 mmol, 1.0 equiv.), silicon oxide (3.76 g) and EtOAc (90 mL) were placed in a reaction vessel equipped with a stirring element. The reaction was carried out for 2.5 hours at room temperature. Silicon oxide was filtered off and solvent was distilled off under reduced pressure, to give the expected product as a yellow oil in 69% yield (2.60 g, 18.5 mmol).

### Alternative Embodiment of Performing Reaction R2

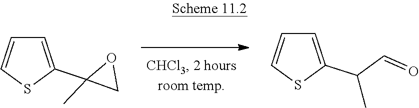

2-Methyl-2-(thiophen-2-yl)epoxide (6.6 g, 47.0 mmol, 1.0 equiv.) and chloroform (60 mL) were placed in a reaction vessel equipped with a stirring element. The reaction was carried out for 2 hours at room temperature. The solvent was distilled off under reduced pressure to give the expected product as a yellow oil in >99% yield (6.59 g, 47.0 mmol).

### Alternative Embodiment of Performing Reaction R2

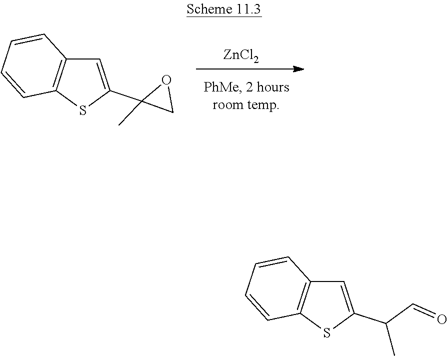

2-Methyl-2-(benzothiophen-2-yl)epoxide (5.32 g, 28.0 mmol, 1 equiv.), zinc chloride (3.81 g, 28.0 mmol, 1 equiv.) and toluene (119 mL) were placed in a reaction vessel equipped with a stirring element. The reaction was carried out for 2 hours at room temperature. The reaction mixture was filtered off on a Schott funnel, the solvent was distilled off under reduced pressure, and the crude product was sublimed under reduced pressure using a Kugelrohr glass oven, to give the expected product as a colorless solid in 97% yield (5.17 g, 27.2 mmol).

### Reaction R3

In step R3 (Scheme 9), the synthesis of an aldehyde with the general formula FX is carried out. For this purpose, an aldehyde of the general formula CX and an alkenyl halide, preferably chloride, a metal hydroxide, preferably NaOH, a quaternary ammonium salt, preferably tetra-N-butylammonium bromide, are used. The transformation is carried out in an organic solvent, preferably toluene. The reaction mixture is carried out at an elevated temperature, preferably 40-60° C. for 30 minutes to 5 hours. The product is separated by extraction from the reaction mixture. It is then dried over a drying agent, preferably sodium sulfate or magnesium sulfate, solid is filtered off, and the solvent is distilled off under reduced pressure. The product is used in the next step without additional purification.

### Embodiment of Performing Reaction R3

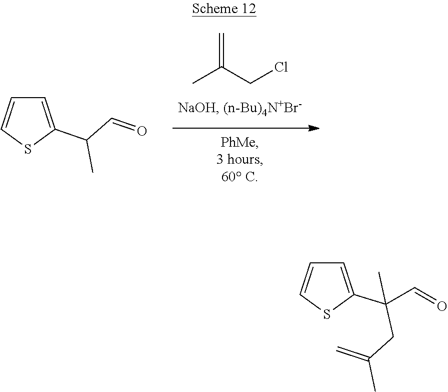

Into a reaction vessel equipped with a stirring element were placed thiophene-2-ylpropanal (6.4 g, 46.0 mmol, 1.00 equiv.), 3-chloro-2-methylpropene (5.1 g, 55.0 mmol, 1.20 equiv.), potassium hydroxide (2.7 g, 69.0 mmol, 1.50 equiv.), tetrabutylammonium bromide (0.6 g, 1.8 mmol, 0.04 equiv.), toluene (60 mL) and water (3 mL). The reaction was carried out for 3 hours at 60° C. The mixture was cooled to room temperature, 15 mL of water was added and extracted with toluene (4×25 mL), dried over sodium sulfate. The drying agent was filtered off and the solvent was distilled off under reduced pressure, to give the expected product as a yellow oil in 68% yield (7.3 g, 31.0 mmol).

TABLE 1
The obtained aldehydes

| | | | ¹H NMR δ (ppm) |
|---|---|---|---|
| | | Molar mass | CHO |
| Structure/Number | Molecular formula | [g/mol] | (solvent) |
| 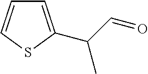 | C₇H₈OS | 140.21 | 9.64 (d, J = 1.7 Hz) (CHCl₃) |
| C1 | | | |
| 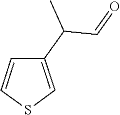 | C₇H₈OS | 140.21 | 9.55 (d, J = 1.8 Hz) (CHCl₃) |
| C2 | | | |
| 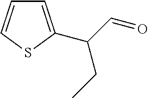 | C₈H₁₀OS | 154.23 | 9.64 (d, J = 3.5 Hz), 9.61 (d, J = 2.4 Hz, 3H) (CHCl₃) |
| C3 | | | |
| 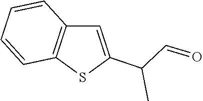 | C₁₁H₁₀OS | 190.27 | 9.73 (d, J = 1.6 Hz) (CHCl₃) |
| C4 | | | |
| 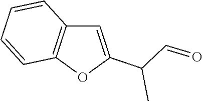 | C₁₁H₁₀O₂ | 174.20 | 9.76 (d, J = 1.4 Hz) (CD₂Cl₂) |
| C5 | | | |
| 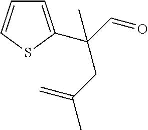 | C₁₁H₁₄OS | 194.30 | 9.52 (s) (CDCl₃) |
| F6 | | | |
| 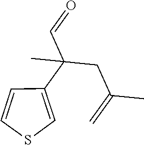 | C₁₁H₁₄OS | 194.30 | 9.52 (s) (CDCl₃) |
| F7 | | | |
| 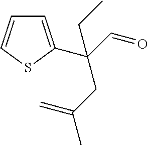 | C₁₂H₁₆OS | 208.32 | 9.50 (s) (CDCl₃) |
| F8 | | | |
| 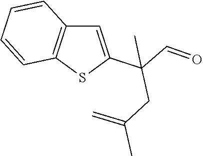 [SMILES: C=C(C)CC(C)(C=O)c1cc2ccccc2s1] | C₁₅H₁₆OS | 244.36 | 9.61 (s) (CDCl₃) |
| F9 | | | |
| 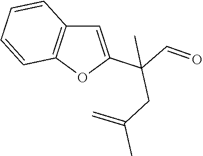 [SMILES: C=C(C)CC(C)(C=O)c1cc2ccccc2o1] | C₁₅H₁₆O₂ | 228.29 | 9.67 (s) (CD₂Cl₂) |
| F10 | | | |

### Reaction R4

In step R4 (Scheme 9), the synthesis of an enol ether of the general formula EX is carried out. For this purpose, DX ketone and a suitable Wittig reagent, preferably chloride, a strong base, preferably potassium tert-butoxide, are used. The transformation is carried out in an anhydrous organic solvent, preferably tetrahydrofuran. The reaction mixture is carried out in the temperature range −78° C.-RT for 16 hours. Then n-heptane is added to precipitate phosphine oxide. After filtering off solids and distilling off the solvent, the product is separated by column chromatography.

### Alternative Embodiment of Performing Reaction R4

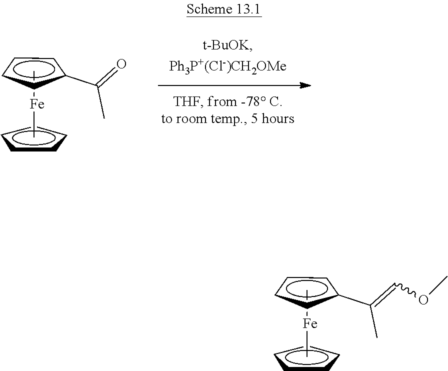

Wittig's reagent (13.2 g, 38.5 mmol, 1.35 equiv.), potassium tert-butoxide (20.4 g, 38.5 mmol, 1.35 equiv.) and anhydrous tetrahydrofuran (57 mL) were placed in a reaction vessel equipped with a stirring element under an argon atmosphere. The reaction was carried out for one hour at −78° C. The mixture was then warmed to room temperature and allowed to stir vigorously for 30 minutes. The mixture was cooled again to −78° C., after which a solution of acetylferrocene (6.50 g, 28.5 mmol, 1 equiv.) in anhydrous tetrahydrofuran (10 mL) was added dropwise. After 30 minutes, the mixture was warmed to room temperature and the reaction was carried out for 16 hours at this temperature. The solvent was then evaporated under reduced pressure, to the residue n-heptane (250 mL) was added, the phosphine oxide was precipitated and filtered off. The solvent was distilled off under reduced pressure, the crude product was purified by column chromatography (Al₂O₃×5% H₂O) collecting fractions using eluent from 0 to 20% ethyl acetate in n-hexane. The expected product was obtained as a red-orange oil with a yield of 96% (6.98 g, 27.2 mmol).

¹H NMR (400 MHz, CDCl₃) δ ppm: 6.22-6.20 (m, 0.53×1H), 5.96-5.94 (m, 0.47×1H), 4.60-4.56 (m, 0.47×2H), 4.25-4.22 (m, 0.53×2H), 4.19-4.16 (m, 0.47×2H), 4.16-4.14 (m, 0.53×2H), 4.12 (s, 0.53×5H), 4.08 (s, 0.47×5H), 3.65 (s, 0.47×3H), 3.64 (s, 0.53×3H), 1.93 (d, J=1.4 Hz, 0.53×3H), 1.84 (d, J=1.4 Hz, 0.47×3H).

¹³C NMR (101 MHz, CDCl₃) δ ppm: 142.8, 141.5, 11.0, 109.3, 87.8, 83.6, 69.1, 68.9, 67.9, 67.7, 67.6, 64.2, 59.9, 59.8, 17.5, 12.8.

### Reaction R5

In step R5 (Scheme 9), the synthesis of an aldehyde of the general formula CX is carried out. For this purpose, an enol ether EX and a suitable inorganic acid, preferably hydrobromic acid, are used. The transformation is carried out in a mixture of organic solvent—water, preferably acetone—water (4-1 v/v). The acid is added dropwise at a temperature below −50° C., using a dry ice-acetone cooling bath, and then the mixture is heated to 45° C. and stirred vigorously for another 2 hours until full conversion of the substrate is achieved. Next, a weak base, preferably sodium bicarbonate, is added until a pH of 8 is obtained, the product is extracted with an organic solvent, preferably methylene chloride, then dried over a drying agent, preferably sodium or magnesium sulfate. After filtering off solids and evaporating the solvent under reduced pressure, the product is obtained.

### Embodiment of Performing Reaction R5

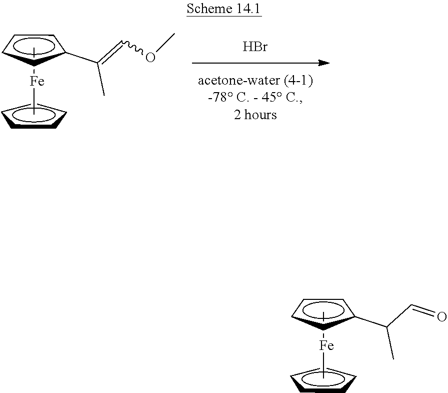

Into a reaction vessel equipped with a stirring element, ether (2.60 g, 10.2 mmol, 1 equiv.), acetone (12 mL) and water (3 mL) were placed. The mixture was cooled below −50° C. and HBr (48% aqueous solution, 2.30 mL, 2 equiv.) was added dropwise under an argon atmosphere. The reaction was then carried out for 2 hours at 45° C. An aqueous solution of NaHCO₃ was added to the reaction mixture until a pH of 8. The product was extracted with methylene chloride, dried over magnesium sulfate, which was then filtered off. The solvent was distilled off under reduced pressure to give the expected product as a maroon oil in 93% yield (2.30 g, 9.50 mmol).

¹H NMR (400 MHz, CDCl₃) δ ppm: 9.72 (d, J=2.2 Hz, 1H), 4.21-4.19 (m, 2H), 4.16 (s, 4H), 4.12-4.09 (m, 2H), 3.26 (qd, J=7.0, 2.3 Hz, 1H), 1.36 (d, J=7.0 Hz, 3H).

¹³C NMR (101 MHz, CDCl₃) δ ppm: 201.0, 85.0, 68.8, 68.3, 68.3, 67.1, 67.0, 45.7, 14.7.

### Example II

### Synthesis of CAAC Ligands

The following Scheme 15 illustrates the synthesis of CAAC ligands allowing the preparation of ruthenium catalysts for olefin metathesis (general formula Ru1a to Ru21a, Scheme 18), which are the subject of the present invention.

The R6 and R8 reactions shown in Scheme 15 were carried out using commercially available compounds based on procedures described in the literature with modifications developed by the authors. Unless otherwise written, the reactions described used commercially available solvents and paid no attention to the presence of oxygen and/or moisture.

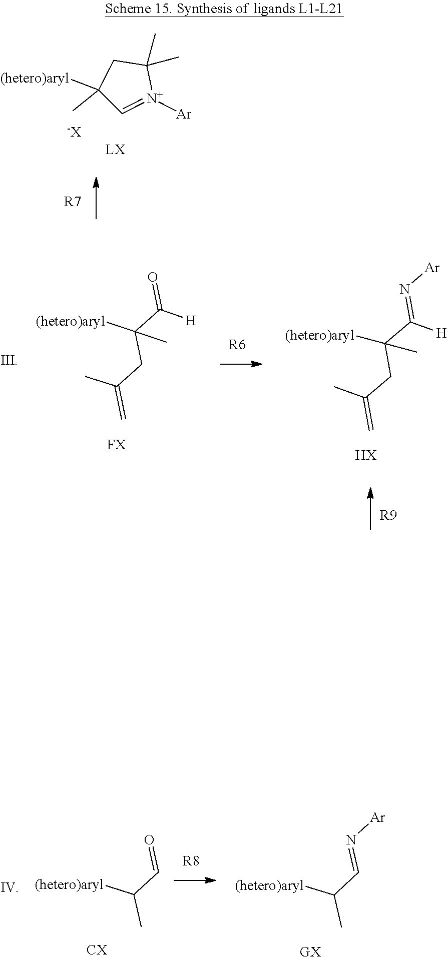

In step R6 (Scheme 15), the synthesis of imine HX is carried out, for this purpose a suitable aniline and an aldehyde of the general formula FX are used, in the presence of an acid, preferably p-toluenesulfonic acid (PTSA). The reaction is carried out preferably in toluene or other organic solvent. The reaction mixture is carried out at the boiling point of the solvent. The product is separated by filtration through neutral Al₂O₃ and distillation of the solvent. The product is used in the next step without additional purification.

In step R7 (Scheme 15), the synthesis of a CAAC ligand of the general formula LX is carried out, for this purpose imine of the general formula HX from step R6 or R9 is used in the presence of an acid, preferably 4 normal hydrochloric acid in dioxane. The reactions are carried out under an argon atmosphere in anhydrous toluene at 85° C. Chloride ions are then exchanged, preferably to tetrafluoroborate ions, and the crude product is precipitated from a mixture of organic solvents, preferably methanol:diethyl ether.

In step R8 (Scheme 15), the synthesis of imine GX is carried out. For this purpose, a suitable aniline and an aldehyde of the general formula CX are used, in the presence of Lewis acid, preferably titanium(IV) isopropanolate. The reaction is carried out in an anhydrous organic solvent, preferably in methanol or another organic solvent at a temperature in the range of 25-45° C. The product is separated by precipitation and filtration through neutral Celite and distillation of the solvent. The product is used in the next step without additional purification.

In step R9 (Scheme 15), the synthesis of imine HX is carried out, and for this purpose imine GX, an alkyl halide, preferably a chloride, a base, preferably n-BuLi, and an organic solvent, preferably tetrahydrofuran (THF) or another organic solvent are used. The reaction is carried out at −78° C.-RT for 10 minutes, cooled to −20° C., the alkyl halide is added, then warmed to room temperature and the reaction is carried out for 16 hours. The product is separated by filtration through neutral Celite and distillation of the solvent. The product is used in the next step without additional purification.

### Embodiment of Performing Reactions R6-R7

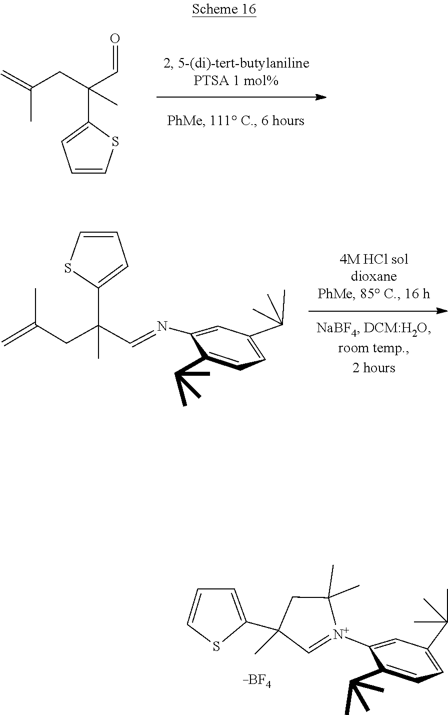

In a round-bottom flask equipped with a stirring element, 2,4-dimethyl-2-thiophenylpent-4-enal (2.11 g, 9.00 mmol, 1.00 equiv.), aniline (1.87 g, 9.00 mmol, 1.00 equiv.) and PTSA (17 mg, 0.09 mmol, 1 mol %) dissolved in PhMe (C=0.30 M) were placed. The reaction was carried out at boiling temperature of toluene until the full conversion of the substrates (collecting water in a Dean-Stark apparatus). The solvent was evaporated under reduced pressure, the crude reaction mixture was dissolved in n-hexane, filtered through neutral alumina (Al₂O₃, neutral, Brockman Grade I), washed with a mixture of n-hexane:ethyl acetate (98:2, v/v), and dried under reduced pressure to give imine in 44% yield (1.50 g, 3.94 mmol), which was used in the next step without further purification.

The imine from the previous step, 4 M HCl (solution in dioxane, 2.53 g, 2.46 mL, 9.83 mmol, 2.5 equiv.) and anhydrous PhMe (C=0.50 M) were placed in a round-bottom flask under an argon atmosphere. The reaction was carried out for 16 hours at 85° C. The solvent was evaporated under reduced pressure. The crude product was dissolved in a methylene chloride water mixture, NaBF₄ (0.86 g, 7.86 mmol, 2.0 equiv.) was added and ion exchange was carried out for 2 h. The organic fraction was collected, washed with water and dried over sodium sulfate. The product was precipitated from the MeOH:Et₂O mixture, to give final product as a colorless crystals in 39% yield (0.72 g, 1.53 mmol).

¹H NMR (400 MHz, CDCl₃): 9.62-9.55 (m, 1H), 8.94 (s, 1H), 7.68-7.62 (m, 1H), 7.56-7.47 (m, 3H), 7.39-7.31 (m, 5H), 7.29-7.27 (m, 1H), 7.16-7.07 (m, 2H), 6.71-6.61 (m, 1H), 3.08 (dd, J=23.3, 13.9 Hz, 2H), 2.72 (dd, J=54.9, 14.0 Hz, 2H), 2.16 (s, 3H), 1.95 (s, 3H), 1.85 (s, 3H), 1.57-1.52 (m, 5H), 1.45 (s, 4H), 1.34 (s, 5H), 1.27 (s, 4H), 1.17 (s, 5H).

¹³C NMR (101 MHz, CDCl₃): δ 184.9, 184.5, 151.0, 150.5, 142.2, 142.0, 142.0, 141.2, 132.1, 131.0, 130.7, 130.6, 129.2, 128.9, 128.6, 127.0, 126.6, 126.3, 125.5, 125.5, 123.1, 82.5, 82.5, 81.9, 81.9, 52.6, 52.4, 50.5, 49.2, 37.7, 37.1, 34.7, 34.5, 33.8, 33.7, 31.0, 30.9, 30.7, 29.4, 29.0, 28.9, 27.2, 26.6.

### Embodiment of Carrying Out Reactions R8-R9-R7

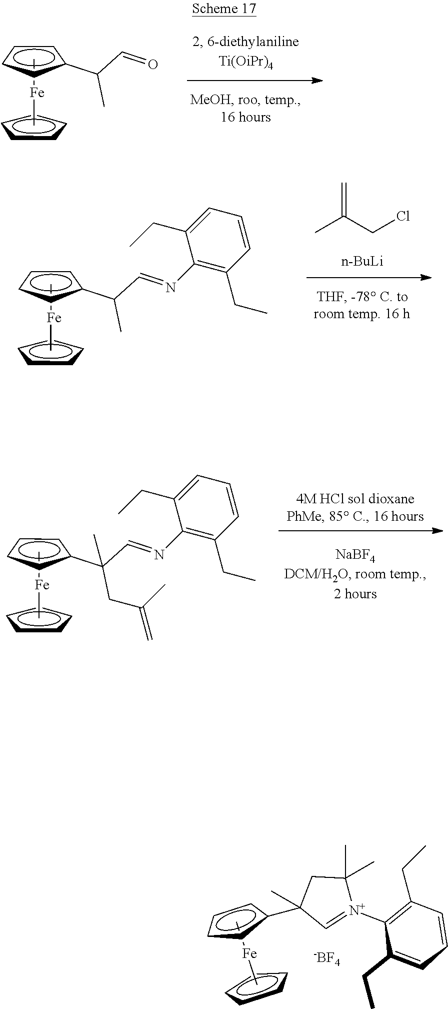

In a round-bottomed flask under an argon atmosphere, 2-ferrocenylpropanal (5.45 g, 22.5 mmol, 1.00 equiv.) and anhydrous methanol (150 mL) were placed, followed by the addition of titanium(IV) isopropanolate (13.3 mL, 45 mmol, 2.00 equiv.). To the reaction mixture thus prepared, 2,6-diethylaniline (4.27 mL, 25.8 mmol, 1.15 equiv.) was added dropwise. The reaction was carried out for 16 h at 45° C. The solvent was evaporated under reduced pressure. The crude product was dissolved in n-pentane and then filtered through neutral Celite. The solvent was evaporated to give the crude product as a maroon oily liquid in 73% yield (6.15 g, 16.5 mmol).

Imine (6.36 g, 17.1 mmol, 1.00 equiv.) and anhydrous tetrahydrofuran (8.55 mL) were placed in a round-bottom flask—previously heated under reduced pressure—under an argon atmosphere. The mixture was then cooled to −78° C. in a dry ice—acetone cooling bath. A solution of n-BuLi (2.35 M solution in n-hexane, 8.72 mL, 20.5 mmol, 1.20 equiv.) was then added dropwise with intense stirring, followed by warming to room temperature. After one hour, the reaction mixture was cooled again to −78° C. in a dry ice-acetone cooling bath and 3-chloro-2-methylpropene (2.50 mL, 25.6 mmol, 1.50 eqn.) was added dropwise. The reaction was carried out for 16 h at room temperature. The solvent was evaporated under reduced pressure. The crude product was purified by distillation to give a maroon oily liquid in 100% yield (7.30 g, 17.1 mmol).

The imine from the previous step (7.30 g, 17.1 mmol, 1 equiv.) dissolved in anhydrous PhMe (C=500 mM) was placed in a round-bottom flask under an argon atmosphere. The mixture was cooled to −78° C. in a dry ice-acetone cooling bath. HCl solution (4 M solution in dioxane, 12.8 mL, 51.2 mmol, 3 equiv.) was then added dropwise and the reaction was carried out for 16 h at 85° C. with vigorous stirring. The solvent was evaporated under reduced pressure. The crude product was dissolved in methylene chloride (about 10 mL), a saturated aqueous solution of NaBF₄ (3.75 g, 34.1 mmol, 2 equiv.) was added and the ion exchange was carried out for 2 h with vigorous stirring. The mixture was extracted three times with methylene chloride, dried with anhydrous magnesium sulfate and filtered through neutral Celite, after which the solvent was evaporated under reduced pressure. The product was precipitated from the DCM:Et₂O mixture to give red crystals in 47% yield (4.14 g, 8.05 mmol).

TABLE 2
Summary of CAAC ligands obtained according to the general procedure from Example III.

| Structure/Number | Selected analytical data |
|---|---|
| 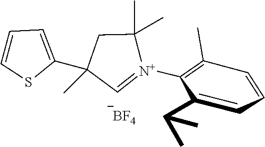 L1 | ¹H NMR (400 MHz, CDCl₃): δ ppm 9.53-9.44 (m, 0.53 × 1H), 9.39-9.27 (m, 0.4 × 1H), 9.25-9.17 (m, 0.07 × 1H) ¹³C NMR (101 MHz, CDCl₃): δ ppm: 186.5, 145.1, 144.8, 134.2, 133.5, 131.7, 131.7, 130.4, 130.3, 130.2, 129.3, 129.1, 129.1, 127.3, 127.0, 126.9, 126.5, 126.4, 126.0, 125.5, 125.1, 84.3, 83.9, 52.8, 52.8, 50.4, 30.1, 29.5, 29.1, 29.0, 29.0, 28.2, 27.9, 27.7, 27.2, 26.1, 26.0, 22.2, 21.9, 19.7, 19.7, 19.2, 18.9, 15.4 |
| 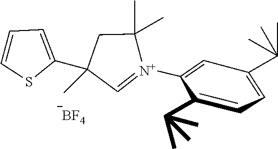 L2 | ¹H NMR (400 MHz, CDCl₃): δ ppm 9.62-9.55 (m, 1H), 8.94 (s, 1H), 7.68-7.62 (m, 1H), 7.56-7.47 (m, 3H), 7.39-7.31 (m, 5H), 7.29-7.27 (m, 1H), 7.16-7.07 (m, 2H), 6.71-6.61 (m, 1H), 3.08 (dd, J = 23.3, 13.9 Hz, 2H), 2.72 (dd, J = 54.9, 14.0 Hz, 2H), 2.16 (s, 3H), 1.95 (s, 3H), 1.85 (s, 3H), 1.57-1.52 (m, 5H), 1.45 (s, 4H), 1.34 (s, 5H), 1.27 (s, 4H), 1.17 (s, 5H); ¹³C NMR (101 MHz, CDCl₃): δ ppm: δ 184.9, 184.5, 151.0, 150.5, 142.2, 142.0, 142.0, 141.2, 132.1, 131.0, 130.7, 130.6, 129.2, 128.9, 128.6, 127.0, 126.6, 126.3, 125.5, 125.5, 123.1, 82.5, 82.5, 81.9, 81.9, 52.6, 52.4, 50.5, 49.2, 37.7, 37.1, 34.7, 34.5, 33.8, 33.7, 31.0, 30.9, 30.7, 29.4, 29.0, 28.9, 27.2, 26.6 |
| 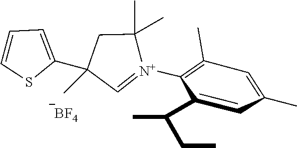 L3 | ¹H NMR (400 MHz, CDCl₃): δ ppm 9.22 (s, 0.20 × 1H), 9.13 (s, 0.28 × 1H), 9.11 (s, 0.22 × 1H), 9.05 (s, 0.30 × 1H) ¹³C NMR (101 MHz, CDCl₃): δ 186.5, 186.3, 186.1, 144.6, 144.5, 143.7, 143.6, 142.4, 142.3, 142.0, 141.9, 141.8, 141.8, 141.6, 133.7, 133.6, 133.2, 133.1, 131.0, 130.9, 130.9, 130.9, 129.2, 129.1, 129.0, 128.5, 128.1, 128.0, 127.4, 127.2, 127.1, 127.1, 126.4, 126.3, 126.3, 126.0, 126.0, 126.0, 125.9, 84.6, 84.2, 83.7, 83.6, 52.6, 52.6, 52.6, 52.5, 50.4, 50.2, 50.1, 50.0, 36.8, 36.8, 36.1, 36.0, 33.4, 33.3, 29.1, 29.0, 29.0, 28.9, 28.8, 28.3, 28.1, 27.9, 27.9, 27.5, 27.5, 27.4, 26.6, 22.6, 22.5, 21.3, 21.3, 21.3, 20.0, 20.0, 19.6, 19.5, 19.2, 19.1, 12.7, 12.5, 12.3, 11.8 |
| 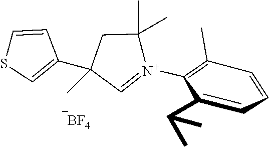 L4 | ¹H NMR (400 MHz, CD₂Cl₂): δ 9.29 (s, 0.5 × 1H), 9.24 (s, 0.5 × 1H), 7.56-7.44 (m, 3H), 7.41-7.37 (m, 0.5 × 1H), 7.35-7.32 (m, 0.5 × 1H), 7.31-7.23 (m, 2H), 3.16 (d, J = 14.0 Hz, 0.5 × 1H), 3.12 (d, J = 14.1 Hz, 0.5 × 1H), 2.75-2.62 (m, 0.5 × 1H), 2.69 (d, J = 14.1 Hz, 0.5 × 1H), 2.66 (d, J = 13.9 Hz, 0.5 × 1H), 2.34 (s, 0.5 × 3H), 2.41-2.26 (m, 0.5 × 1H), 2.07 (s, 0.5 × 3H), 1.95 (s, 0.5 × 3H), 1.89 (s, 0.5 × 3H), 1.62 (s, 0.5 × 3H), 1.57 (s, 0.5 × 3H), 1.45 (s, 0.5 × 3H), 1.42 (s, 0.5 × 3H), 1.40 (d, J = 6.7 Hz, 0.5 × 3H), 1.23 (d, J = 6.7 Hz, 0.5 × 3H), 1.15 (d, J = 6.7 Hz, 0.5 × 3H), 0.95 (d, J = 6.8 Hz, 0.5 × 3H) ¹³C NMR (101 MHz, CDCl₂): δ 187.9, 145.2, 145.1, 140.2, 139.9, 134.3, 133.9, 132.0, 132.0, 131.0, 130.6, 130.5, 129.1, 129.0, 125.8, |
| | 125.7, 125.7, 125.7, 122.9, 122.8, 85.0, 84.6, 53.4, 53.4, 49.2, 49.0, |
| | 30.4, 29.8, 28.7, 28.6, 28.1, 27.9, 27.6, 27.1, 26.1, 26.0, 22.1, 22.1, |
| | 19.8, 19.4 |
| 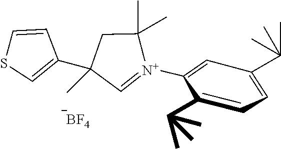 L5 | ¹H NMR (400 MHz, CDCl₃): δ 9.58 (s, 0.57 × 1H), 9.10 (s, 0.43 × 1H), 7.63 (d, J = 8.7 Hz, 0.57 × 1H), 7.55-7.47 (m, 2H), 7.46- 7.40 (m, 1H), 7.38 (dd, J = 3.0, 1.5 Hz, 0.43 × 1H), 7.30 (dd, J = 5.1, 1.5 Hz, 0.57 × 1H), 7.27 (dd, J = 5.0, 1.6 Hz, 0.43 × 1H), 7.22 (d, J = 2.0 Hz, 0.43 × 1H), 6.63 (d, J = 2.1 Hz, 0.57 × 1H), 3.24 (d, J = 14.0 Hz, 0.57 × 1H), 3.07 (d, J = 13.8 Hz, 0.43 × 1H), 2.75 (d, J = 13.7 Hz, 0.43 × 1H), 2.53 (d, J = 14.0 Hz, 0.57 × 1H), 2.01 (s, 0.43 × 3H), 1.83 (s, 0.43 × 3H), 1.82 (s, 0.57 × 3H), 1.52 (s, 0.43 × 3H), 1.51 (s, 0.57 × 3H), 1.44 (s, 0.57 × 3H), 1.42 (s, 0.57 × 9H), 1.32 (s, 0.43 × 9H), 1.25 (s, 0.57 × 9H), 1.12 (s, 0.43 × 9H) ¹³C NMR (101 MHz, CDCl₃): δ 186.1, 185.6, 150.8, 150.4, 141.9, 141.3, 139.5, 139.2, 132.0, 131.1, 130.9, 130.8, 128.8, 128.5, 128.4, |
| | 128.4, 125.8, 125.5, 125.0, 123.1, 122.5, 122.3, 82.5, 81.9, 52.9, 52.7, |
| | 48.7, 47.6, 37.6, 37.1, 34.6, 34.5, 33.7, 33.6, 30.9, 30.9, 30.7, 28.7, |
| | 28.4, 27.8, 27.0, 26.2 |
| 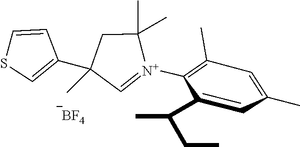 L6 | ¹H NMR (400 MHz, CDCl₃): δ 9.38 (s, 0.23 × 1H), 9.31 (s, 0.27 × 1H), 9.28 (s, 0.23 × 1H), 9.22 (s, 0.23 × 1H), 7.60 (m, 0.23 × 1H + 0.27 × 1H), 7.49 (m, 0.23 × 1H + 0.27 × 1H), 7.45-7.40 (m, 1H), 7.34- 7.26 (m, 1H), 7.04-6.98 (m, 1H), 6.98-6.89 (m, 1H), 3.23 (d, J = 13.9 Hz, 0.27 × 1H), 3.23 (d, J = 14.3 Hz, 0.27 × 1H), 3.22 (d, J = 14.2 Hz, 0.23 × 1H), 3.18 (d, J = 14.2 Hz, 0.23 × 1H), 2.67 (d, J = 14.2 Hz, 0.27 × 1H), 2.63 (d, J = 14.1 Hz, 0.23 × 1H), 2.60 (d, J = 14.2 Hz, 0.27 × 1H), 2.60 (d, J = 14.1 Hz, 0.23 × 1H), 2.39-1.19 (m, 18H), 1.32 (d, J = 6.7 Hz, 0.27 × 3H), 1.16 (d, J = 6.7 Hz, 0.27 × 3H), 1.11 (d, J = 6.7 Hz, 0.23 × 3H), 1.01 (t, J = 7.4 Hz, 0.23 × 3H), 0.85 (d, J = 6.7 Hz, 0.23 × 3H), 0.78 (t, J = 7.4 Hz, 0.27 × 3H), 0.70 (t, J = 7.4 Hz, 0.23 × 3H), |
| | 0.45 (t, J = 7.4 Hz, 0.27 × 3H) |
| | ¹³C NMR (101 MHz, CDCl₃): δ 188.0, 188.0, 187.9, 187.5, 144.5, |
| | 144.3, 143.6, 143.5, 141.8, 141.7, 141.6, 141.5, 139.5, 139.5, 139.3, |
| | 139.3, 133.6, 133.5, 133.1, 132.9, 130.9, 130.9, 130.8, 130.7, 128.8, |
| | 128.7, 128.5, 128.4, 128.4, 128.3, 128.2, 126.1, 126.1, 126.0, 126.0, |
| | 125.8, 125.6, 125.6, 125.6, 122.8, 122.7, 122.6, 84.4, 84.1, 83.7, 83.6, |
| | 53.0, 52.9, 52.9, 48.7, 48.5, 48.1, 47.9, 36.8, 36.8, 36.1, 35.8, 33.3, |
| | 33.3, 28.9, 28.9, 28.7, 28.7, 28.5, 28.4, 28.4, 28.1, 27.4, 27.2, 27.1, |
| | 27.1, 27.0, 25.8, 22.5, 22.4, 21.3, 21.3, 21.3, 19.9, 19.8, 19.6, 19.4, |
| | 19.0, 18.9, 12.6, 12.5, 12.2, 11.9 |
| 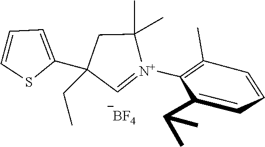 L7 | ¹H NMR (400 MHz, CDCl₃): δ 9.44 (s, 0.5 × 1H), 9.33 (s, 0.5 × 1H), 7.48 (dd, J = 3.7, 1.1 Hz, 0.5 × 1H), 7.46-7.31 (m, 3H), 7.25-7.20 (m, 1H), 7.19-7.15 (m, 0.5 × 1H), 7.14-7.10 (m, 1H), 3.05 (d, J = 14.3 Hz, 1H), 2.79-2.63 (m, 1h + 0.5 × 1H), 2.54-2.42 (m, 0.5 × 1H), 2.42-2.13 (m, 0.5 × 3H + 2H), 2.00 (s, 0.5 × 3H), 1.60 (d, J = 18.8 Hz, 3H), 1.47 (d, J = 8.3 Hz, 3H), 1.37 (d, J = 6.7 Hz, 0.5 × 3H), 1.16 (dd, J = 8.5, 6.7 Hz, 3H), 1.02 (td, J = 7.4, 2.7 Hz, 3H), 0.87 (d, J = 6.7 Hz, 0.5 × 3H); ¹³C NMR (101 MHz, CDCl₃): δ 186.4, 186.0, 144.9, 144.9, 140.4, 140.2, 134.1, 133.6, 131.7, 131.6, 130.5, 130.4, 130.2, 130.1, 129.4, 129.3, 127.8, 127.7, 126.3, 125.8, 125.2, 125.1, 84.0, 83.5, 57.6, 57.5, |
| | 46.9, 46.3, 35.3, 34.4, 30.2, 29.1, 28.7, 27.7, 27.5, 26.8, 26.0, 25.9, |
| | 22.0, 22.0, 19.8, 18.9, 8.7 |
| 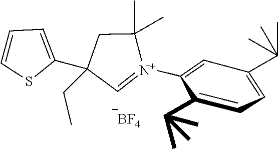 L8 | ¹H NMR (400 MHz, CDCl₃): δ 9.71 (s, 0H), 9.59 (s, 0H), 9.35 (s, 0H), 9.01 (s, 0H), 7.66-7.47 (m, 1H), 7.43-7.26 (m, 1H), 7.16- 7.10 (m, 0H), 6.63-6.55 (m, 0H), 3.08 (d, J = 14.0 Hz, 0H), 2.96- 2.79 (m, 0H), 2.69-2.52 (m, 0H), 2.50-2.25 (m, 0H), 2.07-1.94 (m, 0H), 1.81 (s, 1H), 1.55 (d, J = 4.1 Hz, 1H), 1.49 (s, 1H), 1.44 (s, 2H), 1.33 (s, 1H), 1.25 (s, 2H), 1.11 (s, 2H), 0.92 (td, J = 7.3, 2.4 Hz, 1H); ¹³C NMR (101 MHz, CDCl₃): δ 184.1, 150.8, 150.4, 141.9, 141.4, 139.9, 139.1, 132.0, 131.1, 130.7, 129.3, 129.1, 128.8, 128.6, 127.7, 127.5, 126.3, 125.7, 125.5, 123.3, 82.2, 81.7, 57.2, 57.0, 48.2, 45.1, 37.6, 37.0, 34.7, 34.5, 34.2, 33.7, 33.7, 33.5, 33.1, 33.0, 31.2, 31.0, |
| | 30.9, 29.3, 27.2, 26.5, 8.3, 8.2 |
| 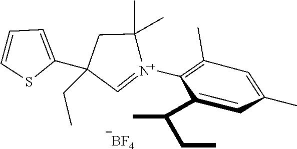 L9 | ¹H NMR (400 MHz, CDCl₃): δ 9.51-9.46 (m, 0.25 × 1H), 9.41- 9.36 (m, 0.28 × 1H), 9.33-9.29 (m, 0.21 × 1H), 9.27-9.22 (m, 0.26 × 1H), ¹³C NMR (101 MHz, CDCl₃): δ 186.7, 186.4, 186.2, 185.8, 144.9, 144.4, 143.8, 143.7, 141.9, 141.8, 141.8, 141.6, 140.4, 140.3, 140.1, 139.8, 133.7, 133.6, 133.4, 133.3, 131.0, 131.0, 130.9, 130.8, 129.7, 129.5, 129.4, 128.5, 128.4, 128.2, 128.1, 128.0, 128.0, 127.9, 126.2, 126.2, 126.2, 126.1, 125.7, 125.7, 83.9, 83.6, 83.1, 83.0, 57.5, 57.4, 57.3, 57.3, 47.1, 46.9, 46.3, 46.1, 37.1, 36.9, 35.8, 35.8, 35.4, 35.3, 34.3, 33.3, 33.3, 29.8, 29.0, 29.0, 28.9, 28.8, 27.8, 27.6, 27.4, 27.3, 27.3, 26.2, 22.6, 22.2, 21.4, 21.3, 19.8, 19.8, 19.7, 19.7, 18.9, 18.8, 12.6, 12.5, 12.2, 11.9, 8.8, 8.7, 8.7, 8.5 |
| 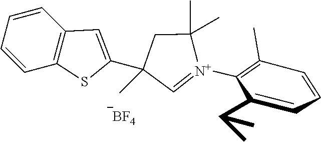 L10 | ¹H NMR (400 MHz, CD₂Cl₂): δ 9.23 (s, 0.5 × 1H), 9.19 (s, 0.5 × 1H), 7.93-7.80 (m, 2H), 7.62 (m, 0.5 × 1H), 7.53-7.47 (m, 0.5 × 1H + 1H), 7.46-7.38 (m, 2H + 0.5 × 1H), 7.38-7.34 (m, 0.5 × 1H), 7.31-7.28 (m, 0.5 × 1H), 7.27-7.24 (m, 0.5 × 1H), 3.18 (d, J = 14.3 Hz, 0.5 × 1H), 3.13 (d, J = 14.2 Hz, 0.5 × 1H), 2.83 (d, J = 14.2 Hz, 0.5 × 1H), 2.82 (d, J = 14.2 Hz, 0.5 × 1H), 2.75-2.57 (m, 1H), 2.35 (s, 0.5 × 3H), 2.19 (s, 0.5 × 3H), 2.13 (s, 0.5 × 3H), 2.07 (s, 0.5 × 3H), 1.66 (s, 0.5 × 3H), 1.61 (s, 0.5 × 3H), 1.56 (s, 0.5 × 3H), 1.55 (s, 0.5 × 3H), 1.41 (d, J = 6.7 Hz, 0.5 × 3H), 1.29 (d, J = 6.7 Hz, 0.5 × 3H), 1.17 (d, J = 6.8 Hz, 0.5 × 3H), 1.00 (d, J = 6.7 Hz, 0.5 × 3H) ¹³C NMR (101 MHz, CD₂Cl₂): δ 186.5, 186.2, 145.1, 144.9, 143.3, |
| | 142.8, 140.3, 140.1, 139.4, 139.2, 134.1, 133.8, 132.1, 132.0, 130.8, |
| | 130.7, 130.5, 130.4, 125.9, 125.8, 125.7, 125.6, 125.6, 125.6, 124.8, |
| | 124.7, 123.7, 123.3, 122.7, 122.6, 85.3, 84.8, 50.4, 50.2, 30.2, 29.8, |
| | 28.6, 28.6, 28.4, 27.7, 27.7, 27.6, 26.2, 26.0, 22.1, 21.9, 19.7, 19.6 |
| 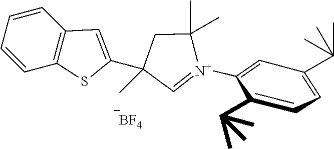 L11 | ¹H NMR (400 MHz, CD₂Cl₂): δ 9.48 (s, 0.6 × 1H), 9.03 (s, 0.4 × 1zH), 7.92-7.80 (m, 2H), 7.72 (d, J = 8.7 Hz, 0.6 × 1H), 7.66- 7.55 (m, 1H + 0.4 × 1H), 7.56 (d, J = 0.7 Hz, 0.6 × 1H), 7.53 (d, J = 0.7 Hz, 0.4 × 1H), 7.46-7.35 (m, 2H), 7.22 (d, J = 2.0 Hz, 0.4 × 1H), 6.78 (d, J = 2.1 Hz, 0.6 × 1H), 3.22 (d, J = 14.2 Hz, 0.6 × 1H), 3.17 (d, J = 13.9 Hz, 0.4 × 1H), 2.87 (d, J = 13.8 Hz, 0.4 × 1H), 2.69 (d, J = 14.2 Hz, 0.6 × 1H), 2.17 (s, 0.4 × 3H), 1.99 (s, 0.6 × 3H), 1.87 (s, 0.4 × 3H), 1.60 (s, 3H), 1.56 (s, 0.6 × 3H), 1.46 (s, 0.6 × 9H), 1.35 (s, 0.4 × 9H), 1.28 (s, 0.6 × 9H), 1.20 (s, 0.4 × 9H) ¹³C NMR (101 MHz, CD₂Cl₂): δ 184.4, 184.3, 151.0, 151.0, 143.1, 142.9, 142.1, 141.8, 140.3, 140.1, 139.6, 139.0, 132.4, 131.7, 131.0, |
| | 130.9, 129.3, 129.1, 125.7, 125.7, 125.5, 125.5, 125.1, 124.7, 124.7, |
| | 124.6, 123.8, 123.4, 123.2, 122.6, 83.6, 82.8, 53.2, 53.2, 50.2, 48.8, |
| | 37.8, 37.4, 34.8, 34.7, 33.8, 30.9, 30.9, 30.8, 29.7, 28.7, 28.5, 27.3, |
| | 26.5 |
| 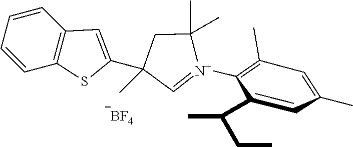 L12 | ¹H NMR (400 MHz, CD₂Cl₂): δ 9.19 (s, 0.22 × 1H), 9.13 (s, 0.25 × 1H), 9.09 (s, 0.53 × 1H) ¹³C NMR (101 MHz, CD₂Cl₂): δ 186.5, 186.3, 186.2, 186.2, 144.7, 144.4, 143.8, 143.7, 143.2, 143.1, 142.9, 142.8, 142.6, 142.5, 142.4, 142.3, 140.3, 140.2, 140.1, 139.3, 139.2, 139.2, 133.7, 133.6, 133.5, 133.3, 131.2, 131.1, 131.1, 131.0, 128.9, 128.5, 128.4, 126.6, 126.6, 126.6, 126.5, 125.8, 125.8, 125.8, 125.8, 125.6, 125.6, 125.5, 125.5, 124.8, 124.8, 124.7, 124.6, 123.8, 123.7, 123.7, 123.7, 122.6, 122.6, 122.6, 85.3, 85.0, 84.6, 84.5, 53.3, 53.2, 50.3, 50.1, 50.1, 50.1, 37.0, 37.0, 36.4, 36.4, 33.5, 33.5, 29.2, 29.0, 28.9, 28.7, 28.6, 28.5, 28.4, 28.3, 27.9, 27.9, 27.5, 27.5, 27.4, 27.1, 22.7, 22.6, 21.3, 21.3, 21.3, |
| | 20.2, 19.9, 19.7, 19.5, 19.5, 19.4, 12.7, 12.5, 12.2, 12.0 |
| 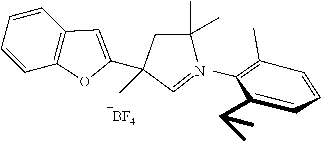 L13 | ¹H NMR (400 MHz, CD₂Cl₂): δ 8.93 (s, 1H), 7.67 (dd, J = 0.8, 1.4 Hz, 0.45 × 1H), 7.66 (dd, J = 0.7, 1.4 Hz, 0.55 × 1H), 7.55-7.46 (m, 2H), 7.43-7.35 (m, 3H), 7.34-7.28 (m, 2H), 7.03 (d, J = 0.9 Hz, 0.45 × 1H), 6.97 (d, J = 0.9 Hz, 0.55 × 1H), 3.16 (d, J = 14.1 Hz, 0.45 × 1H), 3.10 (d, J = 14.0 Hz, 0.55 × 1H), 2.99 (hept, J = 6.7 Hz, 0.55 × 1H), 2.79-2.66 (m, 0.45 × 1H), 2.69 (d, J = 14.1 Hz, 0.45 × 1H), 2.68 (d, J = 14.0 Hz, 0.55 × 1H), 2.36 (s, 0.55 × H), 2.33 (s, 0.45 × H), 2.11 (s, 0.55 × 3H), 2.08 (s, 0.45 × H), 1.71 (s, 0.55 × 3H), 1.69 (s, 0.45 × 3H), 1.67 (s, 0.55 × 3H), 1.67 (s, 0.45 × 3H), 1.41 (d, J = 6.9 Hz, 0.55 × 3H), 1.41 (d, J = 6.7 Hz, 0.45 × 3H), 1.16 (d, J = 6.7 Hz, 0.45 × 3H), 0.96 (d, J = 6.7 Hz, 0.55 × 3H) ppm |
| | ¹³C NMR (101 MHz, CD₂Cl₂): δ 186.2, 185.9, 155.8, 155.8, 154.2, |
| | 153.6, 145.3, 145.2, 134.2, 134.0, 132.3, 132.2, 131.2, 131.0, 130.7, |
| | 128.3, 128.3, 126.2, 126.1, 126.0, 125.7, 124.3, 124.2, 122.5, 122.4, |
| | 111.6, 111.4, 106.3, 106.1, 85.8, 85.3, 52.2, 47.7, 47.4, 30.3, 29.8, |
| | 29.7, 28.5, 28.4, 27.6, 26.3, 26.2, 23.9, 23.0, 22.3, 22.2, 19.9, 19.6 |
| 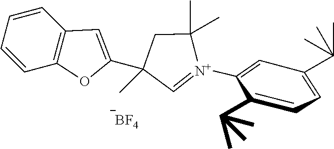 L14 | ¹H NMR (400 MHz, CD₂Cl₂): δ 9.16 (s, 0.47 × 1H), 8.84 (s, 0.53 × 1H), 7.75-7.55 (m, 3H), 7.52 (dq, J = 18.5, 0.9 Hz, 0.47 × 1H), 7.50 (dq, J = 18.4, 0.9 Hz, 0.53 × 1H), 7.38 (m, 1H), 7.30 (m, 1H), 7.22 (d, J = 2.1 Hz, 0.53 × 1H), 7.03 (d, J = 0.9 Hz, 0.47 × 1H), 6.97 (d, J = 0.9 Hz, 0.53 × 1H), 6.89 (d, J = 2.1 Hz, 0.47 × 1H), 3.26 (d, J = 13.9 Hz, 0.47 × 1H), 3.22 (d, J = 13.7 Hz, 0.53 × 1H), 2.69 (d, J = 13.9 Hz, 0.53 × 1H), 2.60 (d, J = 14.1 Hz, 0.47 × 3H), 2.15 (s, 0.53 × 3H), 2.00 (s, 0.47 × 3H), 1.86 (s, 0.53 × 3H), 1.76 (s, 0.47 × 3H), 1.64 (s, 0.53 × 3H), 1.62 (s, 0.47 × 3H), 1.46 (s, 0.47 × 9H), 1.35 (s, 0.53 × 9H), 1.31 (s, 9H) ¹³C NMR (101 MHz, CD₂Cl₂): δ 184.3, 183.9, 155.8, 155.6, 154.0, 153.3, 151.2, 151.1, 142.2, 142.1, 132.6, 131.9, 131.2, 131.1, 129.6, |
| | 129.3, 128.4, 128.3, 126.0, 126.0, 125.5, 124.2, 124.1, 124.0, 122.4, |
| | 122.3, 111.7, 111.7, 106.6, 106.2, 84.2, 83.5, 52.0, 51.9, 47.4, 46.6, |
| | 38.0, 37.5, 35.0, 34.9, 34.0, 33.9, 31.3, 31.1, 31.0, 30.1, 27.5, 27.3, |
| | 24.1, 23.7 |
| 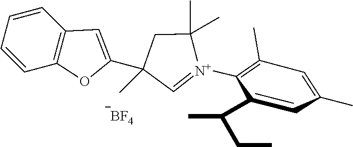 L15 | ¹H NMR (400 MHz, CD₂Cl₂): δ 8.87 (s, 0.4 × H), 8.82 (s, 0.6 × 1H), 7.67 (d, J = 7.7 Hz, 1H), 7.48 (d, J = 8.2 Hz, 1H), 7.38 (td, J = 1.4, 7.8 Hz, 1H), 7.31 (td, J = 1.1, 7.5 Hz, 1H), 7.15-7.08 (m, 2H), 6.99 (d, J = 5.3 Hz, 0.6 × 1H), 6.99 (d, J = 5.3 Hz, 0.4 × 1H), 3.11 (d, J = 13.9 Hz, 0.6 × 1H), 3.11 (d, J = 14.2 Hz, 0.4 × 1H), 2.66 (d, J = 14.0 Hz, 0.6 × 1H), 2.66 (d, J = 14.0 Hz, 0.4 × 1H), 2.66-2.52 (m, 1H), 2.38 (s, 0.6 × 3H), 2.38 (s, 0.4 × 3H), 2.33 (s, 3H), 2.12 (s, 0.6 × 3H), 2.12 (s, 0.4 × 3H), 1.86-1.63 (m, 1H + 3H + 3H), 1.37 (d, J = 6.7 Hz, 0.6 × 3H), 1.46-1.26 (m, 1H), 1.07 (t, J = 7.4 Hz, 0.4 × 3H), 0.92 (d, J = 6.6 Hz, 0.4 × 3H), 0.45 (t, J = 7.4 Hz, 0.6 × 3H) ¹³C NMR (101 MHz, CD₂Cl₂): δ 186.2, 186.1, 155.8, 155.6, 154.1, |
| | 153.8, 144.9, 143.8, 142.6, 142.5, 133.6, 133.5, 131.2, 131.2, 128.2, |
| | 128.2, 126.6, 126.6, 126.0, 124.2, 124.2, 122.4, 111.4, 111.3, 106.4, |
| | 106.1, 85.1, 84.8, 51.9, 51.9, 47.5, 47.3, 36.4, 36.2, 33.8, 29.7, 29.3, |
| | 29.3, 27.7, 27.3, 23.2, 23.0, 22.7, 21.4, 21.4, 20.6, 19.8, 19.7, 12.7, |
| | 11.9 |
| 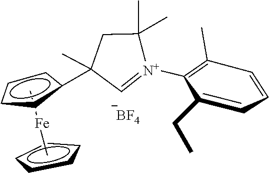 L16 | ¹H NMR (400 MHz, CDCl₃): δ 8.66 (bs, 0.57 × H), 8.59 (bs, 0.43 × 1H), 7.42-7.33 (m, 1H), 7.29 (d, J = 7.6 Hz, 0.57 × 1H), 7.21 (d, J = 7.7 Hz, 1H), 7.16 (d, J = 7.4 Hz, 0.43 × 1H), 4.41-4.26 (m, 8H), 4.19-4.12 (m, 1H), 2.84-2.44 (m, 2H + 0.57 × 2H), 2.37-2.23 (m, 2H × 0.43 + 0.57 × 3H), 2.13 (s, 0.43 × 3H), 2.05-1.96 (m, 3H), 1.64 (s, 0.57 × 3H), 1.63 (s, 0.43 × 3H), 1.56 (s, 0.43 × 3H), 1.54 (s, 0.57 × 3H), 1.26 (t, J = 7.3 Hz, 0.43 × 3H), 1.04 (t, J = 7.3 Hz, 0.57 × 3H) ppm ¹³C NMR (101 MHz, CDCl₃): δ 187.4, 187.4, 140.0, 139.9, 134.1, 133.5, 131.5, 131.5, 131.2, 131.1, 130.3, 129.8, 127.9, 127.8, 89.8, 89.5, 83.9, 83.8, 70.3, 70.3, 69.9, 69.8, 69.4, 69.3, 67.6, 67.2, 66.5, 66.2, 50.8, 50.8, 49.1, 48.9, 29.7, 29.1, 27.9, 27.5, 25.9, 25.6, 25.1, 24.6, 19.6, 19.6, 15.6, 14.8 |
| 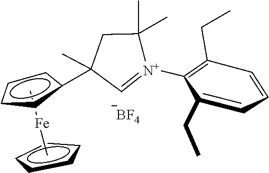 | ¹H NMR (400 MHz, CD₂Cl₂): δ 8.48 (s, 1H), 7.52 (t, J = 7.8 Hz, 1H), 7.39 (d, J = 7.3 Hz, 1H), 7.31 (d, J = 7.7 Hz, 1H), 4.47-4.44 (m, 1H), 4.43-4.40 (m, 1H), 4.36-4.29 (m, 6H), 4.18-4.15 (m, 1H), 2.82 (d, J = 14.1 Hz, 1H), 2.69 (d, J = 14.1 Hz, 1H), 2.64-2.51 (m, 3H), 2.37-2.26 (m, 1H), 2.03 (s, 3H), 1.65 (s, 3H), 1.55 (s, 3H), 1.30 (t, J = 7.5 Hz, 3H), 1.08 (t, J = 7.5 Hz, 3H) ¹³C NMR (101 MHz, CD₂Cl₂): δ 187.0, 140.3, 140.0, 132.1, 128.5, 128.5, 89.8, 84.3, 70.8, 70.5, 69.7, 67.4, 66.7, 51.2, 49.7, 29.7, 28.0, 25.7, 25.6, 25.2, 15.8, 15.2 |
| L17 | |
| 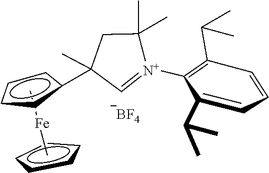 | ¹H NMR (400 MHz, CD₂Cl₂): δ 8.45 (s, 1H), 7.56 (t, J = 7.8 Hz, 1H), 7.41-7.37 (m, 1H), 7.36-7.31 (m, 1H), 4.47-4.43 (m, 1H), 4.43-4.40 (m, 1H), 4.39-4.36 (m, 1H), 4.34 (s, 5H), 4.20-4.14 (m, 1H), 2.86 (d, J = 14.2 Hz, 1H), 2.77-2.65 (m, 2H), 2.53 (hept, J = 7.4 Hz, 1H), 1.99 (s, 3H), 1.67 (s, 3H), 1.55 (s, 3H), 1.38 (d, J = 6.7 Hz, 3H), 1.32 (d, J = 6.8 Hz, 3H), 1.15 (d, J = 6.8 Hz, 3H), 0.87 (d, J = 6.8 Hz, 3H) ¹³C NMR (101 MHz, CD₂Cl₂): δ 186.6, 145.1, 144.9, 132.6, 129.0, 126.2, 125.8, 89.9, 84.2, 70.9, 70.5, 69.8, 67.7, 66.7, 51.1, 49.0, 30.2, 30.1, 29.7, 27.8, 26.5, 26.3, 25.5, 22.6, 21.9 |
| L18 | |
| 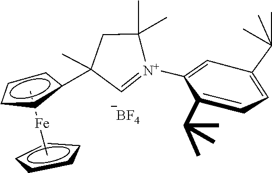 L19 | ¹H NMR (400 MHz, CDCl₃): δ 9.13 (s, 0.24 × 1H), 8.55 (s, 0.76 × 1H) ppm |
| 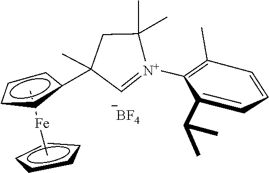 L20 | ¹H NMR (400 MHz, CD₂Cl₂): δ 8.57 (s, 0.56 × 1H), 8.41 (s, 0.44 × 1H), 7.51-7.45 (m, 1H), 7.40-7.36 (m, 0.44 × 1H), 7.35-7.32 (m, 0.56 × 1H), 7.30-7.26 (m, 0.56 × 1H), 7.26-7.22 (m, 0.44 × 1H), 4.46-4.42 (m, 1H), 4.41-4.36 (m, 1H + 0.44 × 1H), 4.32 (s, 0.44 × 5H), 4.32 (s, 0.56 × 5H), 4.26-4.24 (m, 0.56 × 1H), 4.22-4.19 (m, 0.44 × 1H), 4.17-4.15 (m, 0.56 × 1H), 2.86 (d, J = 14.2 Hz, 0.44 × 1H), 2.78 (d, J = 14.1 Hz, 0.56 × 1H), 2.75-2.65 (m, 1H + 0.44 × 1H), 2.59 (hept, J = 6.9 Hz, 0.56 × 1H), 2.33 (s, 0.56 × 3H), 2.16 (s, 0.44 × 3H), 2.01 (s, 0.56 × 3H), 1.99 (s, 0.44 × 3H), 1.67 (s, 0.56 × 3H), 1.65 (s, 0.44 × 3H), 1.59 (s, 0.44 × 3H), 1.55 (s, 0.56 × 3H), 1.39 (d, J = 6.7 Hz, 0.44 × 3H), 1.34 (d, J = 6.8 Hz, 0.56 × 3H), 1.13 (d, J = 6.8 Hz, 0.44 × 3H), 0.89 (d, J = 6.8 Hz, 0.56 × 3H) ¹³C NMR (101 MHz, CD₂Cl₂): δ 187.4, 186.8, 145.2, 145.2, 134.2, |
| | 134.1, 132.1, 132.0, 131.0, 130.8, 130.7, 130.5, 126.0, 125.5, 90.2, |
| | 89.7, 84.6, 84.4, 70.6, 70.6, 70.5, 70.3, 69.7, 69.7, 67.6, 67.1, 67.0, |
| | 66.5, 51.2, 51.1, 49.6, 49.2, 30.1, 30.1, 30.0, 29.1, 28.4, 27.8, 26.4, |
| | 26.2, 25.8, 25.3, 22.5, 21.9, 20.0, 19.9 |
| 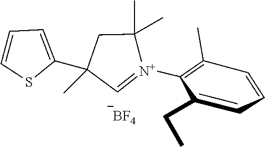 L21 (two diastereoisomers) | ¹H NMR (400 MHz, CDCl₃): δ 9.21 (s, 1H), 7.43-7.31 (m, 4H), 7.20-7.16 (m, 1H), 7.09 (dd, J = 5.2, 3.7 Hz, 1H), 3.08 (d, J = 14.2 Hz, 1H), 2.78 (d, J = 14.3 Hz, 1H), 2.63-2.52 (m, 2H), 2.10 (s, 3H), 2.06 (s, 3H), 1.61 (s, 3H), 1.53 (s, 3H), 1.29 (t, J = 7.5 Hz, 3H) ¹³C NMR (101 MHz, CDCl₃): δ 186.6, 142.1, 140.2, 133.4, 131.4, 130.0, 129.2, 128.0, 127.1, 126.2, 84.2, 52.8, 50.4, 28.8, 28.0, 27.8, 24.9, 19.2, 14.8 ¹H NMR (400 MHz, CDCl₃): δ 9.21 (s, 1H), 7.43-7.36 (m, 2H), 7.33-7.31 (m, 1H), 7.26-7.23 (m, 2H), 7.13-7.10 (m, 1H), 3.06 (d, J = 14.4 Hz, 1H), 2.78 (d, J = 14.2 Hz, 1H), 2.49-2.36 (m, 4H), 2.30-2.19 (m, 1H), 2.12 (s, 3H), 1.65 (s, 3H), 1.63 (s, 3H), 1.07 (t, J = 7.5 Hz, 3H) |
| | ¹³C NMR (101 MHz, CDCl₃): δ 186.6, 142.4, 139.9, 134.3, 131.4, |
| | 130.4, 129.2, 128.0, 127.0, 126.4, 83.9, 52.8, 50.6, 28.8, 28.3, 27.4, |
| | 25.0, 19.6, 15.6 |

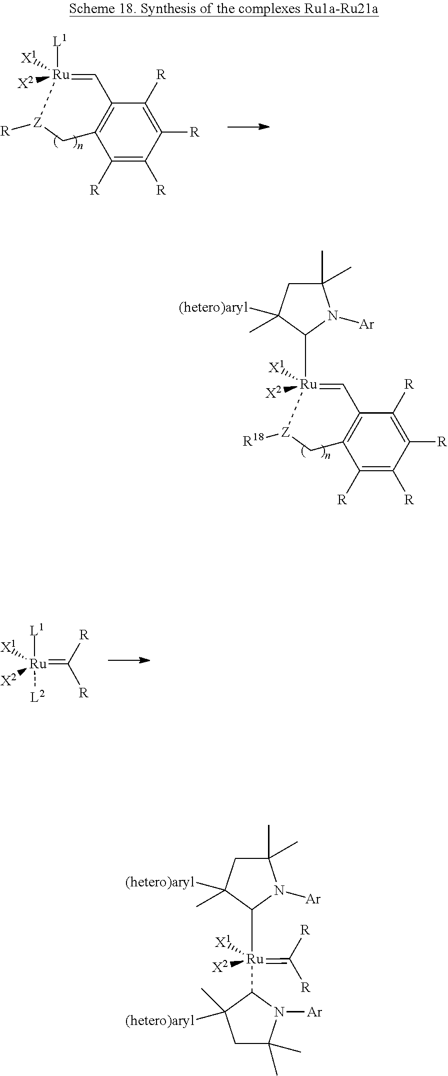

### Example III

### Synthesis of the Complexes Ru1a-Ru20a by Using CAAC Ligands

### Embodiment of the Invention

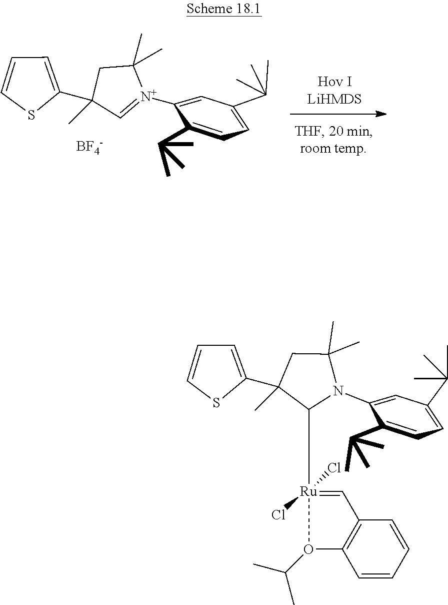

Under an argon atmosphere, the CAAC×BF₄ ligand (430 mg, 916 μmol, 2.20 equiv.), first-generation Hoveyda-Grubbs complex (250 mg, 416 μmol, 1.00 equiv.), and anhydrous THF (C_{c}Ac=0.1 M) were placed in a heated Schlenk vessel and stirred for 1 min. LiHMDS (153 mg, 916 μmol, 2.20 equiv.) was then added and stirred until full conversion was achieved (20 min). The crude mixture was filtered through neutral alumina (Al₂O₃, neutral, Brockman Grade I) with methylene chloride as eluent. The green fraction was collected and evaporated under reduced pressure. A small amount of n-pentane was then added and the mixture was placed in an ultrasonic bath. The product was filtered off and washed with cold n-pentane. The process was repeated twice and then three times using diethyl ether. After drying under vacuum, a green crystalline solid was obtained in 91% yield (267 mg, 380 μmol).

Using the method presented in Example III, a series of complexes from Ru1a to Ru21a were obtained, the structures of which are shown below.

All complexes in the table below were characterized by nuclear magnetic resonance spectroscopy. Table 3 summarizes the benzylidene proton shifts of each complex in the ¹H NMR spectrum in a given solvent.

TABLE 3
Summary of the obtained structures of the ruthenium complexes according to
Example IV general procedure and the shifts of their benzylidene proton in the ¹H NMR spectrum.

| | | | ¹H NMR δ (ppm) |
|---|---|---|---|
| | | Molar mass | Ru = CH |
| Structure/Symbol | Molecular formula | [g/mol] | (solvent) |
| 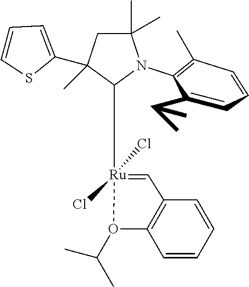 | C₃₁H₃₉Cl₂NORuS | 645.6890 | 18.03, 16.84, 16.54 CDCl₃ |
| Ru1a | | | |
| 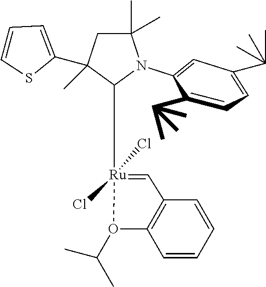 | C₃₅H₄₇Cl₂NORuS | 701.7970 | 17.89 CDCl₃ |
| Ru2a | | | |
| 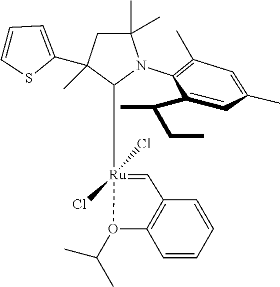 | C₃₃H₄₃Cl₂NORuS | 673.7430 | 16.38, 16.34 CDCl₃ |
| Ru3a | | | |
| 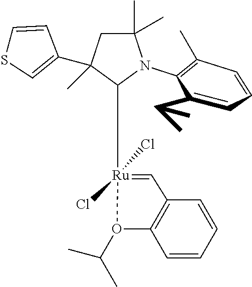 | C₃₁H₃₉Cl₂NORuS | 645.6890 | 16.31, 16.30 CD₂Cl₂ |
| Ru4a | | | |
| 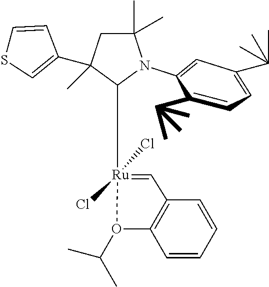 | C₃₅H₄₇Cl₂NORuS | 701.7970 | 17.98, 16.74 CD₂Cl₂ |
| Ru5a | | | |
| 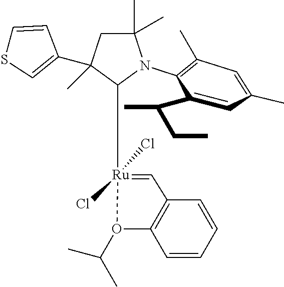 | C₃₃H₄₄Cl₂NORuS | 674.7510 | 16.39, 16.39, 16.35, 16.34 CDCl₃ |
| Ru6a | | | |
| 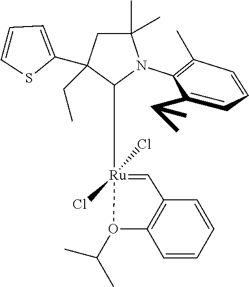 | C₃₂H₄₁Cl₂NORuS | 659.7160 | 16.43, 16.40 CDCl₃ |
| Ru7a | | | |
| 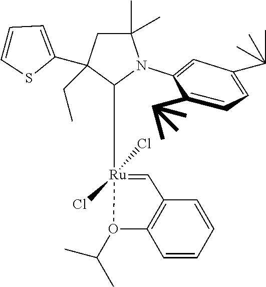 | C₃₆H₄₉Cl₂NORuS | 715.824 | 16.33, 16.32 CDCl₃ |
| Ru8a | | | |
| 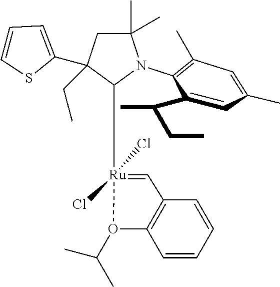 | C₃₄H₄₆Cl₂NORuS | 688.7780 | 16.47, 16.46, 16.42, 16.41 CDCl₃ |
| Ru9a | | | |
| 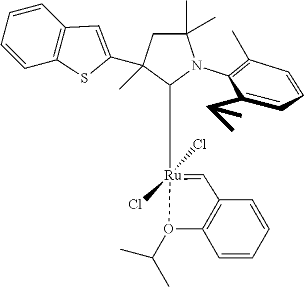 | C₃₅H₄₂Cl₂NORuS | 696.7570 | 16.39, 16.34 CD₂Cl₂ |
| Ru10a | | | |
| 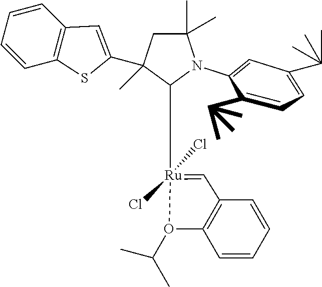 | C₃₉H₄₉Cl₂NORuS | 751.8570 | 18.16, 16.80 CD₂Cl₂ |
| Ru11a | | | |
| 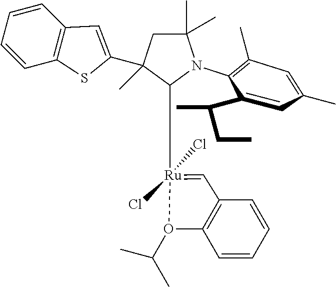 | C₃₇H₄₅Cl₂NORuS | 723.8030 | 16.45, 16.43, 16.40, 16.38 CD₂Cl₂ |
| Ru12a | | | |
| 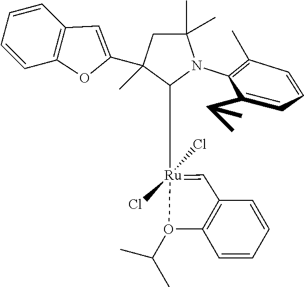 | C₃₅H₄₁Cl₂NO₂Ru | 679.6880 | 16.18, 16.16 CD₂Cl₂ |
| Ru13a | | | |
| 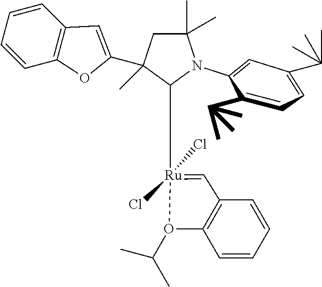 | C₃₉H₄₉Cl₂NO₂Ru | 735.7960 | 18.29, 16.52 CD₂Cl₂ |
| Ru14a | | | |
| 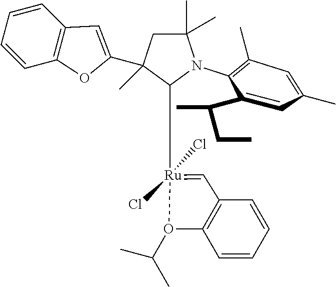 | C₃₇H₄₅Cl₂NO₂Ru | 707.7420 | 16.22, 16.18 CD₂Cl₂ |
| Ru15a | | | |
| 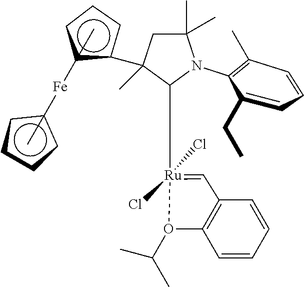 | C₃₆H₄₃Cl₂FeNORu | 733.5610 | 16.23, 16.21 CD₂Cl₂ |
| Ru16a | | | |
| 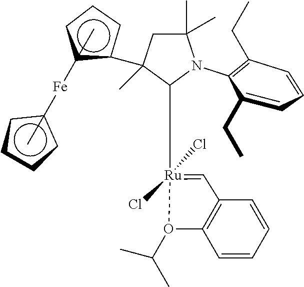 | C₃₇H₄₅Cl₂FeNORu | 747.1271 | 16.36 CDCl₃ |
| Ru17a | | | |
| 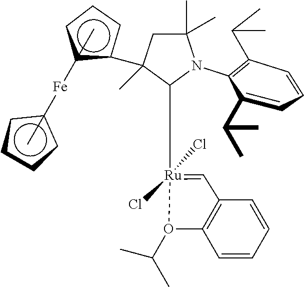 | C₃₉H₄₉Cl₂FeNORu | 775.6420 | 16.46 CDCl₃ |
| Ru18a | | | |
| 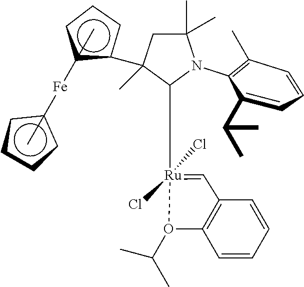 | C₃₇H₄₅Cl₂FeNORu | 747.5880 | 16.26, 16.20 CD₂Cl₂ |
| Ru19a | | | |
| 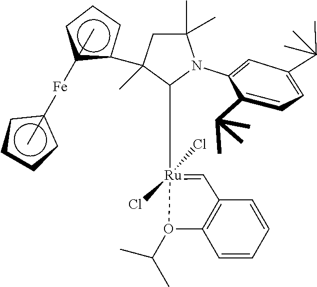 | C₁₄H₅₃Cl₂FeNORu | 803.6960 | 18.44, 16.31 CDCl₃ |
| Ru20a | | | |
| 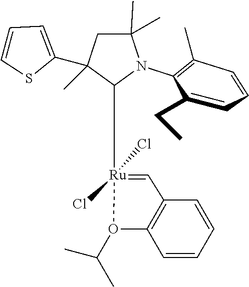 | C₃₀H₃₇Cl₂NORuS | 631.6620 | 16.29, 16.24 CD₂Cl₂ |
| Ru21a | | | |

### Example V

### Study of the Activity of Complexes in the Ethenolysis Reaction of Methyl Oleate

Methyl oleate was degassed by stirring for a minimum of 30 minutes under reduced pressure (oil pump). During this time, a closed Schlenk vessel was prepared and weighed to obtain tare. The oil was then filtered through a syringe filter into the Schlenk vessel and the vessel was weighed together with the oil, thus obtaining the weight of the oil. The vessel with the substrate was subjected to reduced pressure. In a separate vessel, a catalyst solution (˜3 mg in 2 mL of degassed toluene) was prepared. A preheated glass insert for the Amar reactor equipped with a stirring element was placed in the Amar reactor immersed in a preheated oil bath. The reactor was closed and the gas inside was evacuated using an oil pump. A portion of the catalyst (10 ppm or 3 ppm or 500 ppb) was added to the Schlenk vessel containing the oil and the mixture was immediately transferred into the reactor using a Teflon tube. The reactor was filled with ethylene to a dynamic pressure of 10 bar, then stirring was started and the reaction was run for 3 hours or 6 hours. The glovebox and inert gas atmosphere (argon) were not used during the reaction.

After that time, pressure was normalized and the autoclave was disassembled. SnatchCat metal scavenger (scavenger) solution was immediately added and the contents of the vessel were stirred for several minutes. A sample was then taken and, after dilution with toluene, subjected to GC analysis.

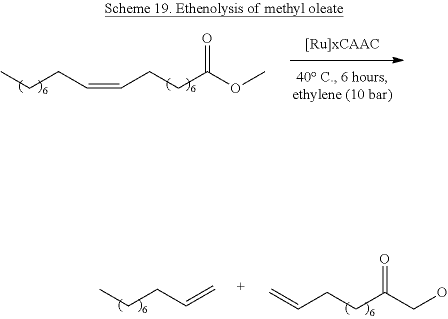

The results of the model reaction are shown in Table 2.

TABLE 4
Comparison of the selectivity of the new ruthenium complexes and ruthenium complexes
known from the literature in the process of ethenolysis

| | | Ethenolysis results | | |
|---|---|---|---|---|
| No. | Structure | Loading [ppm] | TON | Selectivity |
| 1. | 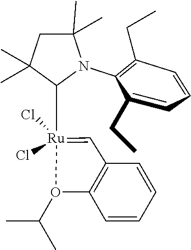 | 100 10 | 5,300¹ 35,000¹ | 73% 83% |
| | Literature state-of-the-art | | | |
| 2. | 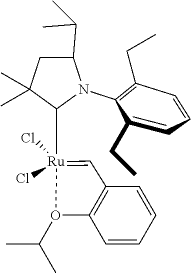 | 100 10 | 5,800² 26,000² | 96% 97% |
| | Literature state-of-the-art | | | |
| 3. | 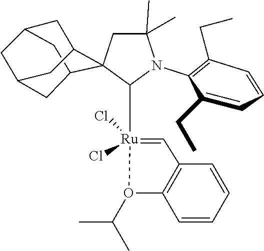 | 3 | —³ | — |
| | Literature state-of-the-art | | | |
| 4. | 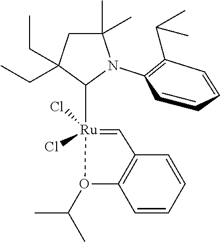 | 3 | 47,000³ | 63% |
| | Literature state-of-the-art | | | |
| 5. | 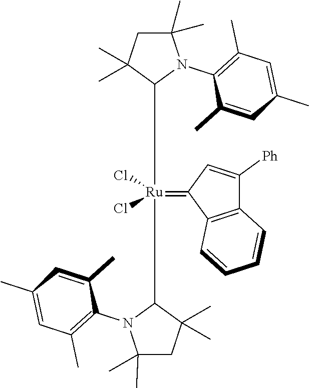 | 10 | —⁴ | — |
| | Literature state-of-the-art | | | |
| 6. | 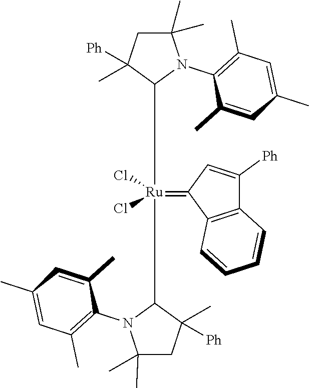 | 10 | 19,000⁴ | 92% |
| | Literature state-of-the-art | | | |
| 7. | 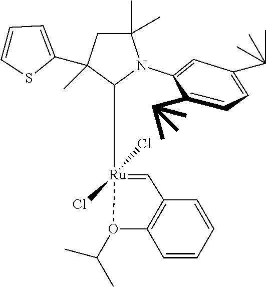 | 3 0.5 | 113,000 101,000 | 88% 88% |
| | Ru2a | | | |
| 8. | 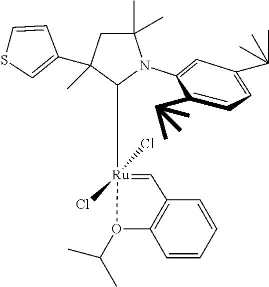 | 3 0.5 | 189,000 255,000 | 88% 88% |
| | Ru5a | | | |
| 9. | 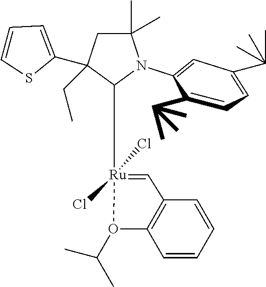 | 3 0.5 | 48,000 57,000 | 98% 96% |
| | Ru8a | | | |
| 10. | 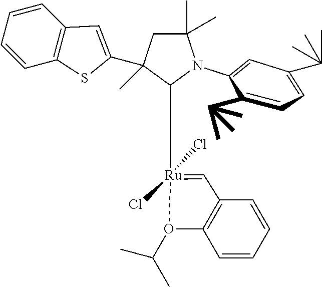 | 3 0.5 | 136,629 182,700 | 88% 88% |
| | Ru11a | | | |
| 11. | 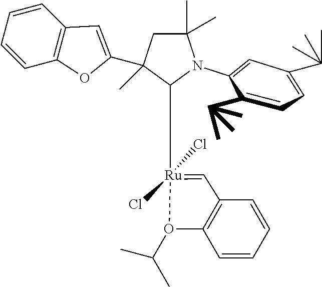 | 3 0.5 | 87,612 130,656 | 88% 81% |
| | Ru14a | | | |
| 12. | 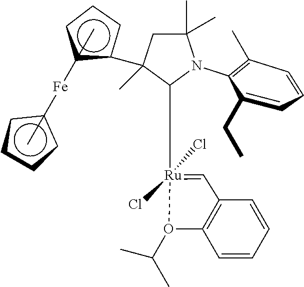 | 10 3 | 52,713 99,006 | 92% 94% |
| | Ru16a | | | |
| 13. | 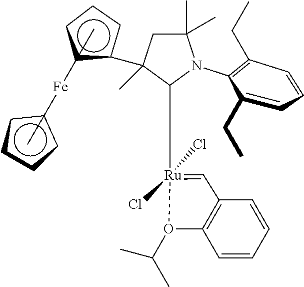 | 10 3 | 65,363 94,668 | 92% 95% |
| | Ru17a | | | |
| 14. | 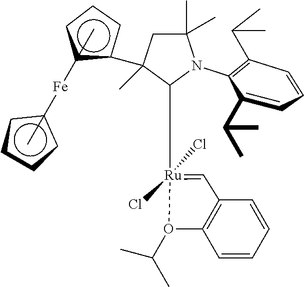 | 10 3 | 2,507 3,187 | 99% 100% |
| | Ru18a | | | |
| 15. | 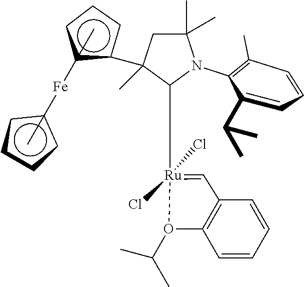 | 10 (isomer ratio = 1:1) 3 (isomer ratio = 1:1) | 40,447 69,881 | 95% 97% |
| | Ru19a | | | |
| 16. | 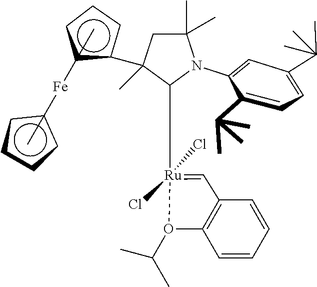 | 10 3 0.5 | 20,297 28,953 34,280 | 49% 54% 39% |
| | Ru20a | | | |
| 17. | 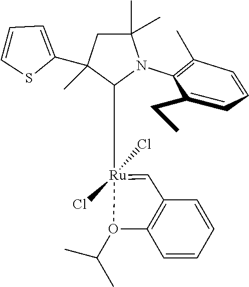 | 10 3 | 44,000 90,000 | 94% 96% |
| | Ru21a | | | |

Example 1: ethylene 99.9%, <30 min, 40° C., pressure of 10 bar
Example 2: 4 hours, 40° C., pressure of 10 bar
Examples 3 and 4: ethylene 99.95%, 3 hours, 40° C., pressure of 10 bar
Examples 5 and 6: ethylene, 99.99%, 4 hours, 40° C., pressure of 10 bar
Examples 7-17: reactions for 10 ppm: ethylene 99.95%, 3 hours, 40° C.; reactions for 3 ppm:
ethylene 99.95%, 3-6 hours, 40° C.; reactions for 0.5 ppm: ethylene 99.995%, 6 hours, 40° C.
(ethylene pressure of 10 bar)
¹Grubbs et al. Organometallics 2008, 27, 563-566.
²Zhang et al. Chem. Commun., 2013, 49, 9491-9493.
³Bertrandt et al. Angew. Chem. Int. Ed., 2015, 54, 1919-1923.
⁴Gawin et al. Angew. Chem. Int. Ed. 2017, 56, 981-986.

### Example VI

Testing the activity of the ruthenium complex in the ethenolysis reaction of methyl oleate outside the glovebox under conditions that do not require an inert gas protective atmosphere (FIG. 3). FIG. 3 shows A) the autoclave assembled in air, B) the open autoclave before the ethenolysis reaction.

The corresponding ethenolysis reactions described in Example V were performed outside the glovebox in an autoclave apparatus placed under the fume hood and located in the air—the reaction system shown in FIG. 3. Subsequent reaction steps were performed in the air without an inert gas protective atmosphere. Ethylene of different purities was used during the reaction, with the purity of ethylene being lower than that described in previous literature reports. Using the new ruthenium complexes Ru1a-Ru22a was observed to be resistant to the use of lower quality ethylene, which did not impair the reaction results as determined by TON and selectivity.

### Example VII

### Synthesis of a CAAC Ligand and a Spiro Carbon Catalyst

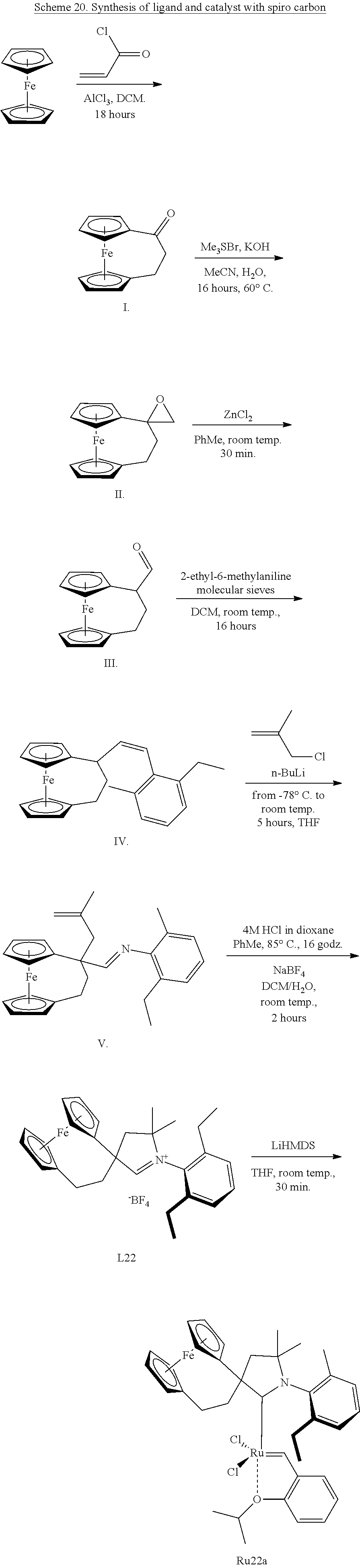

### Synthesis of Compound I

Aluminium(III) chloride (14.3 g, 105 mmol, 1.00 equiv.) and anhydrous DCM (100 mL) were placed under an argon atmosphere in a three-neck flask equipped with a stirring element, and the suspension was cooled to −40° C. In a two-neck flask under an argon atmosphere equipped with a stirring element, ferrocene (20.0 g, 105 mmol, 1.00 equiv.) was dissolved in anhydrous DCM (155 mL) and added dropwise into the aluminum(III) chloride suspension in DCM at −40° C. with stirring. The mixture was then cooled to −78° C. and acryloyl chloride (9.81 mL, 116 mmol, 1.10 equiv.) was added dropwise for 30 min at −78° C. The reaction was carried out for 18 hours at −78° C., then the contents of the flask were poured into water at 0° C. The organic layer was separated, washed with brine, dried over MgSO₄, the drying agent was filtered through neutral Celite and the solvent was evaporated. The crude product was purified by column chromatography (Al₂O₃×5% H₂O) to give the product as an orange solid (7.99 g, 33.3 mmol, 31%).

¹H NMR (400 MHz, CDCl₃): δ 4.87-4.79 (m, 2H), 4.66-4.56 (m, 2H), 4.43-4.30 (m, 2H), 4.10-3.94 (m, 2H), 3.06-2.86 (m, 4H).

¹³C NMR (101 MHz, CDCl₃): δ 212.0, 88.2, 74.1, 72.8, 71.2, 70.4, 69.4, 44.3, 31.9.

### Synthesis of Compound II

Ketone I (1.16 g, 4.83 mmol, 1.0 equiv.), Me₃SBr (0.98 g, 6.28 mmol, 1.3 equiv.), KOH (0.70 g, 12.6 mmol, 2.6 equiv.), distilled water (0.25 mL), and MeCN (10 mL) were placed in a reaction vessel equipped with a stirring element under an argon atmosphere. The contents of the vessel were stirred for 16 h at 60° C. After completion of the reaction, 5 mL of diethyl ether (Et₂O) was added, the precipitate was filtered off, and the precipitate was washed again with diethyl ether (10 mL), the solvent was evaporated under reduced pressure. The residue was washed with diethyl ether (10 mL), the solvent evaporated and washed with n-hexane (10 mL), the solvent evaporated under reduced pressure to give the product as an orange solid (1.02 g, 4.01 mmol, 83%).

¹H NMR (400 MHz, CDCl₃): δ 4.44-4.35 (m, 1H), 4.25-4.22 (m, 1H), 4.19-4.16 (m, 2H), 4.10-4.04 (m, 4H), 2.95 (d, J=5.5 Hz, 1H), 2.90 (dd, J=0.6, 5.4 Hz, 1H), 2.33-2.16 (m, 3H), 2.09-1.99 (m, 1H).

¹³C NMR (101 MHz, CDCl₃): δ 86.6, 81.8, 70.9, 69.1, 68.9, 68.8, 68.7, 68.5, 68.4, 67.7, 56.4, 55.0, 42.5, 22.9.

### Synthesis of Compound III

Epoxide II (0.94 g, 3.68 mmol, 1 equiv.), zinc(II) chloride (0.50 g, 3.68 mmol, 1 equiv.) and toluene (16 mL) were placed in a reaction vessel equipped with a stirring element. The reaction was carried out for 2 hours at room temperature. The reaction mixture was filtered off on a Schott funnel, the solvent was distilled off under reduced pressure to give the product as a bright orange solid (0.77 g, 3.04 mmol, 82%).

¹H NMR (400 MHz, CDCl₃): δ 9.82 (d, J=1.5 Hz, 1H), 4.22-4.19 (m, 1H), 4.18-4.15 (m, 1H), 4.14-4.11 (m, 3H), 4.10-4.07 (m, 1H), 4.06-4.01 (m, 2H), 2.82-2.74 (m, 1H), 2.54-2.39 (m, 2H), 2.05-1.93 (m, 1H), 1.86-1.72 (m, 1H).

¹³C NMR (101 MHz, CDCl₃): δ 201.4, 86.4, 81.4, 71.5, 71.4, 70.0, 69.8, 68.9, 68.1, 68.0, 67.3, 50.4, 35.8, 23.9.

### Synthesis of Compound IV

In a round-bottom flask—previously heated under reduced pressure—under an argon atmosphere, aldehyde III (0.7 g, 2.76 mmol, 1.0 equiv.), 2-ethyl-6-methylaniline (0.38 g, 2.76 mmol, 1.0 equiv.), 4 Å molecular sieves (0.7 g), and methylene chloride (5.5 mL) were placed. The reaction was carried out for 16 h at room temperature. The molecular sieves were filtered off, the solvent was evaporated under reduced pressure to give the product as an orange solid. (0.56 g, 1.49 mmol, 54%).

¹H NMR (400 MHz, CD₂Cl₂): δ 7.75 (d, J=4.7 Hz, 1H), 7.05-6.96 (m, 2H), 6.91 (t, J=7.5 Hz, 1H), 4.22-4.18 (m, 1H), 4.16-4.12 (m, 2H), 4.13-4.05 (m, 4H), 4.06-4.00 (m, 1H), 3.08-2.94 (m, 1H), 2.57-2.47 (m, 2H), 2.43 (q, J=7.5 Hz, 2H), 2.30-2.16 (m, 1H), 2.04 (s, 3H), 2.01-1.90 (m, 1H), 1.08 (t, J=7.5 Hz, 3H).

¹³C NMR (101 MHz, CD₂Cl₂): δ 169.2, 151.0, 133.5, 128.2, 127.0, 126.6, 123.7, 86.9, 85.2, 71.6, 71.0, 69.6, 68.5, 68.2, 68.1, 67.2, 43.9, 39.2, 24.9, 24.4, 18.5, 14.9.

### Synthesis of Compound V

Imine IV (0.55 g, 1.47 mmol, 1.00 equiv.) and anhydrous tetrahydrofuran (3.0 mL) were placed in a round-bottom flask—previously heated under reduced pressure—under an argon atmosphere. The mixture was then cooled to −78° C. in a dry ice-acetone cooling bath. A solution of n-BuLi (2.30 M solution in n-hexane, 0.77 mL, 1.76 mmol, 1.20 equiv.) was then added dropwise with intense stirring, followed by heating to room temperature. After one hour, the reaction mixture was cooled again to −78° C. in a dry ice-acetone cooling bath and 3-chloro-2-methylpropene (0.22 mL, 2.2 mmol, 1.50 equiv.) was added dropwise. The reaction was carried out for 16 h at room temperature. The solvent was evaporated under reduced pressure. The crude product was filtered through a syringe filter to give the product as a red oil. (0.51 g, 1.19 mmol, 80%).

¹H NMR (400 MHz, CDCl₃): δ 7.84 (s, 1H), 7.10-7.03 (m, 1H), 7.06-6.99 (m, 1H), 7.00-6.92 (m, 1H), 4.85-4.80 (m, 1H), 4.80-4.75 (m, 1H), 4.25-4.18 (m, 2H), 4.16-4.11 (m, 3H), 4.11-4.05 (m, 1H), 4.08-4.03 (m, 2H), 2.76 (d, J=14.5 Hz, 1H), 2.70-2.64 (m, 1H), 2.58 (d, J=14.5 Hz, 1H), 2.51 (qd, J=1.6, 7.5 Hz, 2H), 2.43-2.32 (m, 2H), 2.24-2.19 (m, 1H), 2.15 (s, 3H), 1.69 (s, 3H), 1.16 (t, J=7.5 Hz, 3H).

¹³C NMR (101 MHz, CDCl₃): δ 171.3, 150.4, 142.0, 133.3, 128.2, 127.0, 126.3, 123.7, 115.3, 89.6, 87.4, 68.8, 68.8, 68.5, 68.5, 68.4, 68.4, 67.7, 67.7, 46.2, 42.9, 29.8, 25.1, 24.6, 20.6, 19.1, 14.9.

### Synthesis of Compound L22

The imine from the previous step (0.49 g, 1.15 mmol, 1 equiv.) dissolved in anhydrous PhMe (C=500 mM) was placed in a round-bottom flask under an argon atmosphere. The mixture was cooled to −78° C. in a dry ice-acetone cooling bath. HCl solution (0.7 mL, 4 M solution in dioxane, 2.88 mmol, 2.5 equiv.) was then added dropwise and the reaction was carried out for 16 h at 85° C. with vigorous stirring. The solvent was evaporated under reduced pressure. The crude product was dissolved in methylene chloride (about 5 mL), a saturated aqueous solution of NaBF₄ (0.25 g, 2.30 mmol, 2 equiv.) was added and the ion exchange was carried out for 2 h with vigorous stirring. The mixture was extracted three times with methylene chloride, dried with anhydrous magnesium sulfate and filtered through neutral Celite, after which the solvent was evaporated under reduced pressure. The product was precipitated from the DCM:Et₂O mixture to give the product as a light brown solid (0.27 g, 0.53 mmol, 45%).

### Synthesis of Compound Ru22a

Under an argon atmosphere, the CAAC×BF₄ ligand L22 (174 mg, 340 μmol, 1.20 equiv.), the first-generation Hoveyda-Grubbs complex (170 mg, 283 μmol, 1.00 equiv.) and anhydrous THF (C_{CAAC}=0.1 M) were placed in a preheated Schlenk flask and stirred for 1 min. LiHMDS (56 mg, 340 μmol, 1.20 equiv.) was then added and stirred until full conversion was achieved (5 min). After this time, copper(I) chloride (56 mg, 566 μmol, 2.00 equiv.) was added. The crude mixture was filtered through neutral alumina (Al₂O₃, neutral, Brockman Grade I) with methylene chloride as eluent. The green fraction was collected and the solvent was evaporated under reduced pressure. A small amount of n-pentane was then added and the mixture was placed in an ultrasonic bath. The product was drained and washed with cold n-pentane. The process was repeated twice and then using diethyl ether. After drying under vacuum, a green crystalline solid was obtained (164 mg, 220 μmol, 77%).

| Structure/Number | Selected analytical data | | |
|---|---|---|---|
| 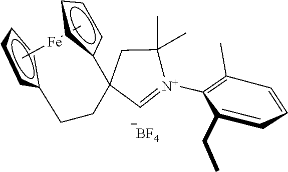 | ¹H NMR (400 MHz, CD₂Cl₂): δ 9.09 (s, 1H), 7.49-7.40 (m, 1H), 7.35 (d, J = 7.7 Hz, 0.62 × 1H), 7.27 (d, J = 7.2 Hz, 0.38 × 1H), 7.25 (d, J = 7.3 Hz, 0.38 × 1H), 7.20 (d, J = 7.5 Hz, 0.62 × 1H), 4.57-4.52 (m, 0.62 × 1H), 4.52-4.48 (m, 0.38 × 1H), 4.40-4.35 (m, 1H), 4.28-4.24 (m, 3H), 4.23-4.19 (m, 1H), 4.19-4.16 (m, 1H), 4.15-4.11 (m, 1H), 2.80 (d, J = 13.9 Hz, 0.62 × 1H), 2.79 (d, J = 13.9 Hz, 0.38 × 1H), 2.70- 2.39 (m, 1H + 2 × 0.31 × 2H + 1H + 2H), 2.33 (s, 0.38 × 3H), 2.31-2.22 (m, 0.19 × 2H), 2.09-1.99 (m, 0.19 × 2H), 1.96 (s, 0.62 × 3H), 1.94-1.88 (m, 1H), 1.58 (s, 0.38 × 3H), 1.55 (s, 0.62 × 3H), 1.50 (s, 0.62 × 3H), 1.48 (s, 0.38 × 3H), 1.26 (t, J = 7.5 Hz, 0.62 × 3H), 0.99 (t, J = 7.5 Hz, 0.38 × 3H); ¹³C NMR (101 MHz, Methylene chloride-d₂): δ | | |
| L22 | 189.7, 189.7, 140.5, 140.4, 134.3, 133.9, 131.9, 131.8, 131.4, 131.4, | | |
| | 130.2, 130.1, 128.3, 128.0, 86.3, 86.0, 84.6, 84.5, 84.4, 84.1, 72.0, 71.8, | | |
| | 71.8, 71.1, 69.9, 69.8, 69.3, 69.2, 69.1, 68.5, 68.4, 67.7, 67.5, 51.2, 51.1, | | |
| | 46.0, 45.7, 45.0, 44.8, 28.4, 27.6, 26.9, 25.5, 24.6, 19.7, 19.3, 19.2, 18.9, | | |
| | 15.6, 15.0 | | |
| | | | ¹H NMR δ (ppm) |
| | | Molar mass | Ru = CH |
| Structure/Symbol | Molecular formula | [g/mol] | (solvent) |
| 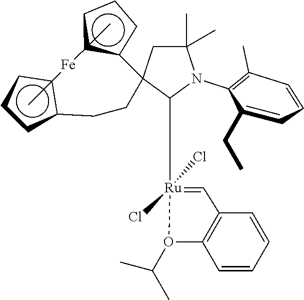 | C₃₇H₄₃Cl₂FeNORu | 745.1115 | 16.40, 16.36 CDCl₃ |
| Ru22a | | | |

1. A precursor of cyclic alkylamine carbenes (CAAC) of the formula CAAC-1 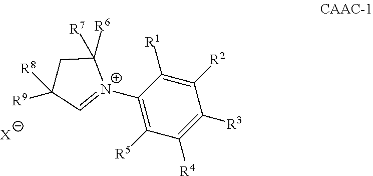
wherein X denotes an anion selected from the group comprising halogen atom, BF₄⁻, PF₆⁻, ClO₄⁻, CF₃SO₂O⁻;
R¹, R², R³, R⁴ and R⁵ independently denote a hydrogen atom, a C₁-C₁₂ alkyl group, a C₃-C₁₂ cycloalkyl group, a C₅-C₂₀ aryl group or a C₅-C₂₀ heteroaryl group, a C₅-C₂₅ aralkyl group, which may be substituted independently by one and/or more substituents selected from the group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxyl group, a C₅-C₂₄ aryloxyl group, a C₅-C₂₀ heteroaryloxyl group, a sulfide (—SR″) group, an amino (—NR″₂) group, in which the R′ group independently denotes a hydrogen atom, C₁-C₅ alkyl, C₆-C₂₄ aryl, C₇-C₂₄ aralkyl, alternatively R¹, R², R³, R⁴ and R⁵ are connected to form a C₅-C₂₅ ring;
each substituent R⁶, R⁷, and R⁸ denotes a hydrogen atom, a halogen atom, a C₁-C₁₂ alkyl group, or a C₅-C₂₀ aryl group, which may be substituted independently by one and/or more substituents selected from the hydrogen-containing group, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxyl group, a C₅-C₂₄ aryloxyl group, a C₅-C₂₀ heteroaryloxyl or a halogen atom, a sulfide group (—SR″), an amino group (—NR″₂), in which the R″ group independently denotes a hydrogen atom, C₁-C₅ alkyl, C₆-C₂₄ aryl, C₇-C₂₄ aralkyl;
R⁹ substituent means a substituted or unsubstituted heterocyclic group or an organometallic complex group selected from thiophene, benzothiophene, furan, benzofuran or ferrocene, which can be substituted independently by one and/or more substituents selected from the group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxyl group, a C₅-C₂₄ aryloxyl group, a C₅-C₂₀ heteroaryloxyl group, a sulfide group (—SR″), an amino group (—NR″₂), wherein the R″ group independently denotes a hydrogen atom, C₁-C₅ alkyl, C₆-C₂₄ aryl, C₇-C₂₄ aralkyl.

2-3. (canceled)

4. The precursor of the formula CAAC-1 according to claim 1, having the structure represented by the formula L1, L2, L3, L4, L5, L6, L7, L8, L9, L10, L11, L12, L13, L14, L15, L16, L17, L18, L19, L20, L21 or L22:

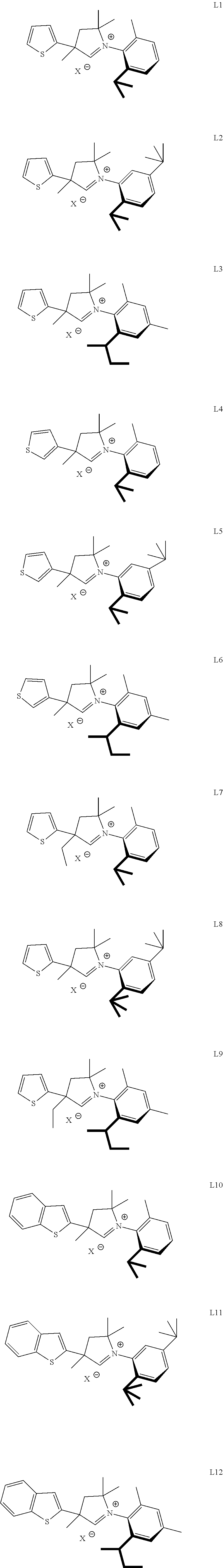

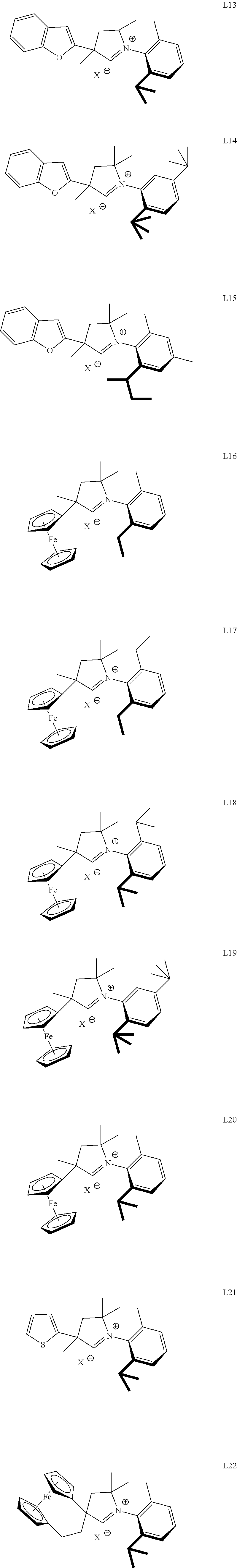

5. A ruthenium complex of formula 1-Ru 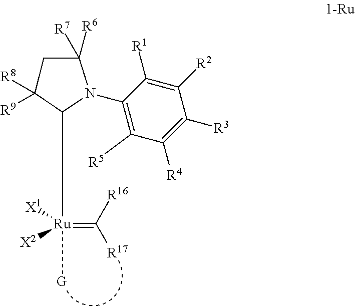
wherein:
X¹ and X² independently of each other denote an anionic ligand selected from the group comprising a halogen anion, a —CN, —SCN, —OR^{a}, —SR^{a}, —O(C═O)R^{a}, —O(SO₂)R^{a} and —OSi(R^{a})₃ group, wherein R^{a} denotes C₁-C₁₂ alkyl, C₃-C₁₂ cycloalkyl, C₂-C₁₂ alkenyl or C₅-C₂₀ aryl, which is optionally substituted with at least one C₁-C₁₂ alkyl, C₁-C₁₂ perfluoroalkyl, C₁-C₁₂ alkoxyl, C₅-C₂₄ aryloxyl, C₅-C₂₀ heteroaryloxyl or a halogen atom;
R¹, R², R³, R⁴ and R⁵ denote independently a hydrogen atom, a C₁-C₁₂ alkyl group, a C₃-C₁₂ cycloalkyl group, a C₅-C₂₀ aryl group or a C₅-C₂₀ heteroaryl group, a C₅-C₂₅ aralkyl group, which may be independently substituted with one and/or more substituents selected from the group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxyl group, a C₅-C₂₄ aryloxyl group, a C₅-C₂₀ heteroaryloxyl group, a sulfide group (—SR″), an amino group (—NR″₂), in which the R″ group independently denotes a hydrogen atom, C₁-C₅ alkyl, C₆-C₂₄ aryl, C₇-C₂₄ aralkyl,
alternatively R¹, R², R³, R⁴ and R⁵ are bonded with each other to form a C₅-C₂₅ ring;
each substituent R⁶, R⁷, and R⁸ denotes a hydrogen atom, a halogen atom, a C₁-C₁₂ alkyl group, or a C₅-C₂₀ aryl group, which may be substituted independently with one and/or more substitutes selected from the group comprising a hydrogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, C₁-C₁₂ alkoxyl group, a C₅-C₂₄ aryloxyl group, a C₅-C₂₀ heteroaryloxyl group or halogen atom, a sulfide group (—SR″), an amino group (—NR″₂), in which the group R″ independently denotes a hydrogen atom, C₁-C₅ alkyl, C₆-C₂₄ aryl, C₇-C₂₄ aralkyl;
R⁹ substituent denotes a substituted or unsubstituted heterocyclic group and/or organometallic complex group selected from thiophene, benzothiophene, furan, benzofuran or ferrocene, which may be substituted independently with one and/or more substituents selected from the group comprising a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxyl group, a C₅-C₂₄ aryloxyl group, a C₅-C₂₀ heteroaryloxyl group, a sulfide group (—SR″), an amino group (—NR″₂), in which the R″ group denotes independently a hydrogen atom, C₁-C₅ alkyl, C₆-C₂₄ aryl, C₇-C₂₄ aralkyl,
R¹⁶ and R¹⁷ denote independently a hydrogen atom, a halogen atom, C₁-C₂₅ alkyl, C₃-C₂₅ cycloalkyl, C₁-C₁₂ perfluoroalkyl, C₂-C₂₅ alkene, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₅ aryl, C₅-C₂₅ aryloxyl, C₆-C₂₅ arylalkyl, C₅-C₂₅ heteroaryl, C₅-C₂₅ heteroaryloxyl, C₅-C₂₅ perfluoroaryl, a 3-12-membered heterocycle comprising a sulfur, oxygen, nitrogen, selenium or a phosphorus atom;
whereby the substituents R¹⁶ and R¹⁷ may be linked together to form a ring selected from the group comprising C₃-C₂₅ cycloalkyl, C₃-C₂₅ cycloalkenyl, C₃-C₂₅ cycloalkynyl, C₅-C₂₅ aryl, C₅-C₂₅ heteroaryl, C₅-C₂₅ perfluoroaryl, a 3-12-membered heterocycle comprising a sulfur, oxygen, nitrogen, selenium or a phosphorus atom, which may be substituted independently by one or more substituents selected from the group comprising a hydrogen atom, a halogen atom, C₁-C₂₅ alkyl, C₃-C₂₅ cycloalkyl, C₇-C₁₂ perfluoroalkyl, C₂-C₂₅ alkene, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₅ aryl, C₅-C₂₅ aryloxyl, C₆-C₂₅ arylalkyl, C₅-C₂₅ heteroaryl, C₅-C₂₅ heteroaryloxyl, C₅-C₂₅ perfluoroaryl, a 3-12-membered heterocycle;
G is selected from such as
ligand with the formula CAAC-1 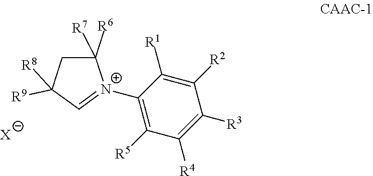
in which X and the substituents R¹ to R⁹ have the above defined meanings
either
heteroatom 1
selected from the group comprising an oxygen atom, a sulfur atom, a selenium atom, substituted by a group selected from such as a hydrogen atom, a fluorine atom, an oxygen atom, C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, a 3-12-membered heterocycle, optionally substituted with an acyl group (—COR′), a cyano group (—CN), a carboxyl group (—COOH), an ester group (—COOR′), an ester group (—CH₂COOR′), an ester group (—CHR′COOR′), an ester group (—C(R′)₂COOR′), an amide group (—CONR′₂), a Weinreb-type amide (—CON(R′)(OR′)), a sulfone group (—SO₂R′), a formyl group (—CHO), a sulfonamide group (—SO₂NR′₂), a ketone group (—COR′), a thioamide group (—CSNR′₂), a thioketone (—CSR′), a thionoester group (—CSOR′), a thioester group (—COSR′), a dithioester group (—CS₂R′), in which the group R′ denotes independently C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl, C₅-C₂₄ heteroaryloxyl and then the dashed line denotes a direct bond between a heteroatom and the substituent R¹⁷ or denotes a bond between the substituent R¹⁷ and a heteroatom via a methylene bridge —CH₂—, —CHR′—, or —CR′₂— wherein the substituent R¹⁴ is C₅-C₁₅ aryl, optionally substituted with 1-4 substituents, independently selected from a group comprising a hydrogen atom, a halogen atom, C₁-C₂₅ alkyl, C₃-C₂₅ cycloalkyl, C₂-C₂₅ alkenyl, C₃-C₂₅ cycloalkenyl, C₂-C₂₅ alkynyl, C₃-C₂₅ cycloalkynyl, C₁-C₂₅ perfluoroalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₆-C₂₀ heteroaryl or C₅-C₂₄ heteroaryloxyl, a 3-12-membered heterocycle, an alkoxyl group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″3), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes C₁-C₅ alkyl, C₁-C₅ perfluoroalkyl, C₆-C₂₄ aryl, C₇-C₂₄ aralkyl, C₅-C₂₄ perfluoroaryl, or
heteroatom 2
selected from a group comprising a nitrogen atom or a phosphorus atom, substituted with the group selected from such as a hydrogen atom, methylidene, optionally substituted with the substituent R′, C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, a 3-12-membered heterocycle, an acyl group (—COR′), an ester group (—COOR′), a tert-butyl butyloxycarbonyl group (t-Boc) or a 9-fluorenylmethoxycarbonyla group (Fmoc), a carbamine group (—CONR′₂), a sulfone group (—SO₂R′), a formyl group (—CHO), in which the R′ group denotes C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, optionally substituted with an acyl group (—COR′), a cyano group (—CN), a carboxyl group (—COOH), an ester group (—COOR′), an ester group (—CH₂COOR′), an ester group (—CHR′COOR′), an ester group (—C(R′)₂COOR′), an amide group (—CONR′₂), a sulfone group (—SO₂R′), a formyl group (—CHO), a sulfonamide group (—SO₂NR′₂), a ketone group (—COR′), a thioamide group (—CSNR′₂), a thioketone group (—CSR′), a thionoester group (—CSOR′), a thioester group (—COSR′), a dithioester group (—CS₂R′), in which the R′ group denotes C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, and then the dashed line denotes a direct bond between the heteroatom and the substituent R¹⁴ or it denotes a bond between the substituent R¹⁷ with a heteroatom via methylene bridge (CH₂)—, —(CHR′)—, or —(CR′₂)—; wherein the substituent R¹⁷ is C₅-C₁₅ aryl, optionally substituted with 1-4 substituents independently selected from the group comprising a hydrogen atom, a halogen atom, C₁-C₂₅ alkyl, C₃-C₂₅ cycloalkyl, C₂-C₂₅ alkenyl, C₃-C₂₅ cycloalkenyl, C₂-C₂₅ alkynyl, C₃-C₂₅ cycloalkynyl, C₁-C₂₅ perfluoroalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₆-C₂₀ heteroaryl or C₅-C₂₄ heteroaryloxyl, a 3-12-membered heterocycle, an alkoxyl group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the group R″ denotes C₁-C₅ alkyl, C₁-C₅ perfluoroalkyl, C₆-C₂₄ aryl, C₇-C₂₄ aralkyl, C₅-C₂₄ perfluoroaryl, or
heteroatom 3
selected from a group comprising a halogen atom and then the dashed line denotes a direct bond between the heteroatom and the R¹⁷ substituent, wherein the R¹⁷ substituent is C₅-C₁₅ aryl, or C₅-C₂₅ polyaryl, optionally substituted with 1-4 substituents selected independently from a group comprising a hydrogen atom, a halogen atom, C₁-C₂₅ alkyl, C₃-C₂₅ cycloalkyl, C₂-C₂₅ alkenyl, C₃-C₂₅ cycloalkenyl, C₂-C₂₅ alkynyl, C₃-C₂₅ cycloalkynyl, C₁-C₂₅ perfluoroalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₆-C₂₀ heteroaryl or C₅-C₂₄ heteroaryloxyl, a 3-12-membered heterocycle, an alkoxy group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes C₁-C₅ alkyl, C₁-C₅ perfluoroalkyl, C₆-C₂₄ aryl, C₇-C₂₄ aralkyl, C₅-C₂₄ perfluoroaryl.

6. The ruthenium complex according to claim 5 represented by the formula 1a-Ru 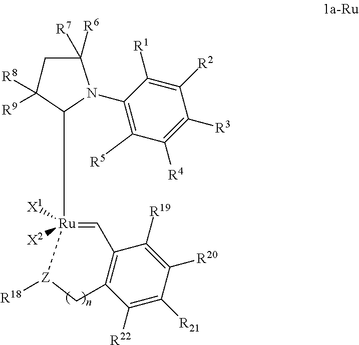
in which X¹ and X² and the R¹, R², R³, R⁴, R⁵, R⁶, R⁷, R⁸, R⁹ substituents have the meanings defined above
‘n’ means 1 or 0
Z is selected from a group comprising halogen atoms, O atom, S atom, Se atom, or a NR′″ group, in which R′″ denotes methylidene, C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl, a 3-12-membered heterocycle, an acyl group (—COR′), an ester group (—COOR′), a tert-butyloxycarbonyl group (t-Boc) or a 9-fluorenylmethoxycarbonyl group (Fmoc), a carbamine group (—CONR′₂), a sulfone group (—SO₂R′), a formyl group (—CHO), in which the R′ group denotes C₁-C₂₅ alkyl, C₁-C₂₅ perfluoroalkyl, C₃-C₂₅ cycloalkyl, C₅-C₂₀ alkoxyl, C₅-C₂₀ aryl, C₅-C₂₀ perfluoroaryl, C₇-C₂₀ aralkyl, C₅-C₂₄ aryloxyl, C₂-C₁₂ alkenyl, C₆-C₂₀ heteroaryl or C₅-C₂₄ heteroaryloxyl,
or a halogen atom, wherein if Z denotes a halogen atom, R¹⁸ is absent;
R¹⁸ means independently a hydrogen atom, C₁-C₂₅ alkyl, C₁-C₂₅ cycloalkyl, C₅-C₂₀ alkoxyl, C₅-C₂₀ aryl, C₅-C₂₄ aryloxyl, a —COOR′″ group, a —CH₂COOR′″ group, a —CONR′″₂ group, a —CH₂CONR′″₂ group, a —COR′″ group, a —CH₂COR′″ group, a —CON(OR′″)(R′″) group, a —CH₂CON(OR′″)(R′″) group or a halogen atom, wherein R′″ denotes C₁-C₁₂ alkyl, C₃-C₁₂ cycloalkyl, C₂-C₁₂ alkenyl, C₆-C₂₀ aryl, which are optionally substituted with at least one C₁-C₁₂ alkyl, C₁-C₁₂ perfluoroalkyl, C₁-C₁₂ alkoxyl, C₆-C₂₄ aryloxyl, or a halogen atom;
R¹⁹, R²⁰, R²¹, and R²² denote independently a hydrogen atom, a halogen atom, a C₁-C₂₅ alkyl group, a C₂-C₂₅ alkenyl group, a C₅-C₂₅ aryl group, an alkoxy group (—OR″), a sulfide group (—SR″), a sulfoxide group (—S(O)R″), a sulfonium group (—S⁺R″₂), a sulfone group (—SO₂R″), a sulfonamide group (—SO₂NR″₂), an amino group (—NR″₂), an ammonium group (—N⁺R″₃), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″₃), a carboxyl group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes C₁-C₅ alkyl, C₁-C₅ perfluoroalkyl, C₆-C₂₄ aryl, C₇-C₂₄ aralkyl, C₅-C₂₄ perfluoroaryl, wherein the R¹⁶, R¹⁷, R¹⁸ and R¹⁹ substituents may be connected, thus forming a substituted or unsubstituted C₄-C₁₀ cyclic or C₄-C₁₂ polycyclic system.

7. The ruthenium complex according to claim 5 of the formula 1b-Ru 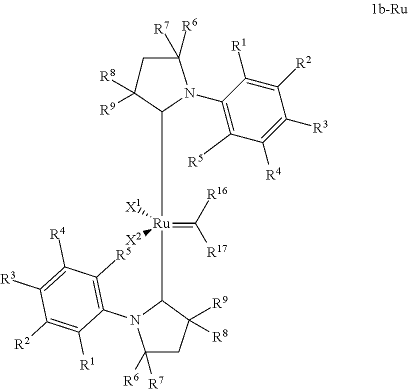
wherein X¹ and X² and the substituents R¹, R², R³, R⁴, R⁵, R⁶, R⁷, R⁸, R⁹ have the above defined meanings
R¹⁶ and R¹⁷ denote independently a hydrogen atom, a halogen atom, C₁-C₂₅ alkyl, optionally substituted; C₃-C₂₅ cycloalkyl, optionally substituted; C₁-C₁₂ perfluoroalkyl, optionally substituted; C₂-C₂₅ alkene, optionally substituted; C₂-C₂₅ alkenyl, optionally substituted; C₃-C₂₅ cycloalkenyl, optionally substituted; C₂-C₂₅ alkynyl, optionally substituted; C₃-C₂₅ cycloalkyl, optionally substituted; C₁-C₂₅ alkoxyl, optionally substituted; C₅-C₂₅ aryl, optionally substituted; C₅-C₂₅ aryloxyl, optionally substituted; C₆-C₂₅ arylalkyl, optionally substituted; C₅-C₂₅ heteroaryl, optionally substituted; C₅-C₂₅ heteroaryloxyl, optionally substituted; C₅-C₂₅ perfluoroaryl, optionally substituted; a 3-12-membered heterocycle comprising a sulfur, oxygen, nitrogen, selenium or a phosphorus atom, optionally substituted;
wherein the R¹⁶ and R¹⁷ substituents may be connected, forming a ring selected from a group comprising C₃-C₂₅ cycloalkyl, C₃-C₂₅ cycloalkenyl, C₃-C₂₅ cycloalkynyl, C₅-C₂₅ aryl, C₅-C₂₅ heteroaryl, C₅-C₂₅ perfluoroaryl, a 3-12-membered heterocycle containing a sulfur, oxygen, nitrogen, selenium or phosphorus atom, which may be independently substituted with one or more substituents selected from a group comprising a hydrogen atom, a halogen atom, C₁-C₂₅ alkyl, C₃-C₂₅ cycloalkyl, C₁-C₁₂ perfluoroalkyl, C₂-C₂₅ alkene, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₅ aryl, C₅-C₂₅ aryloxyl, C₆-C₂₅ arylalkyl, C₅-C₂₅ heteroaryl, C₅-C₂₅ heteroaryloxyl, C₅-C₂₅ perfluoroaryl, a 3-12-membered heterocycle.

8. The ruthenium complex according to claim 5, which is selected from the complexes represented by the formulae of Ru1a, Ru2a, Ru3a, Ru4a, Ru5a, Ru6a, Ru7a, Ru8a, Ru9a, Ru10a, Ru11a, Ru11a, Ru12a, Ru13a, Ru14a, Ru15a, Ru16a, Ru17a, Ru18a, Ru19a, Ru20a, Ru21a, Ru22a:

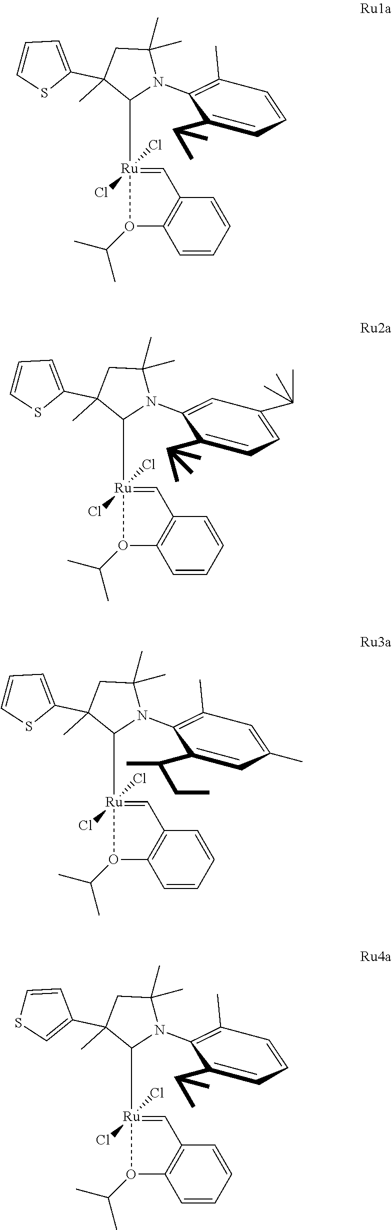

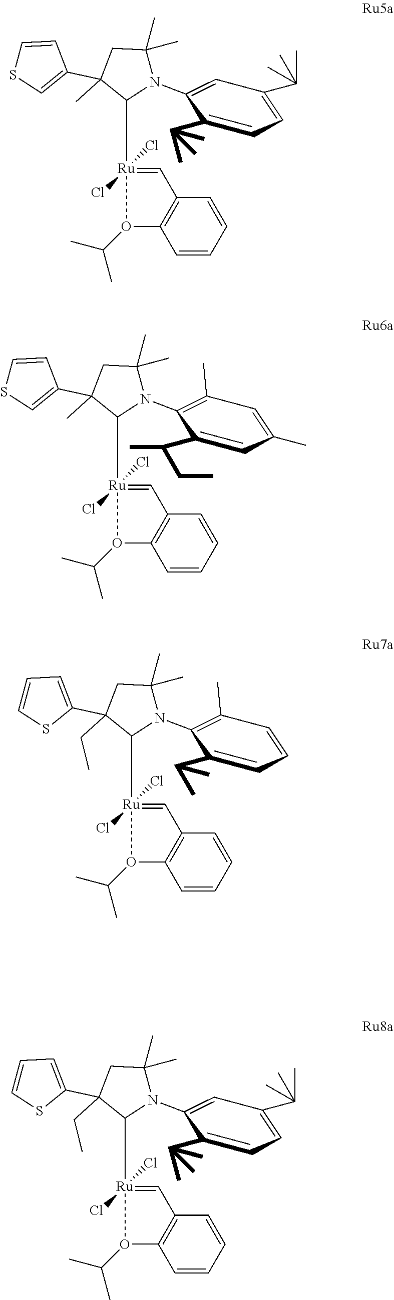

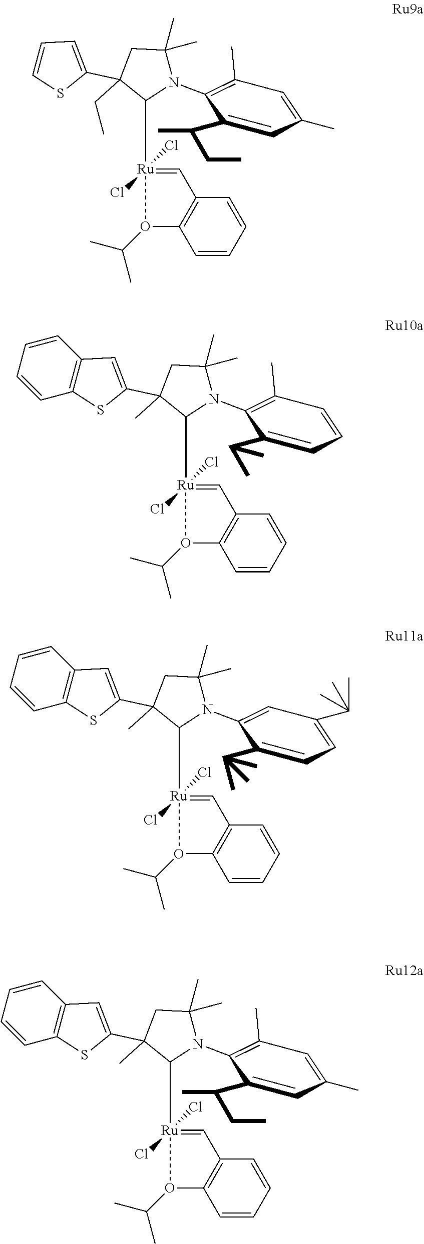

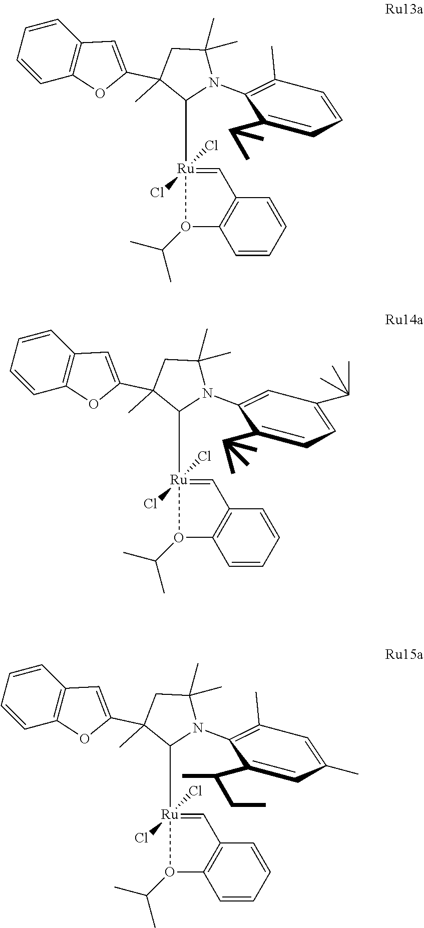

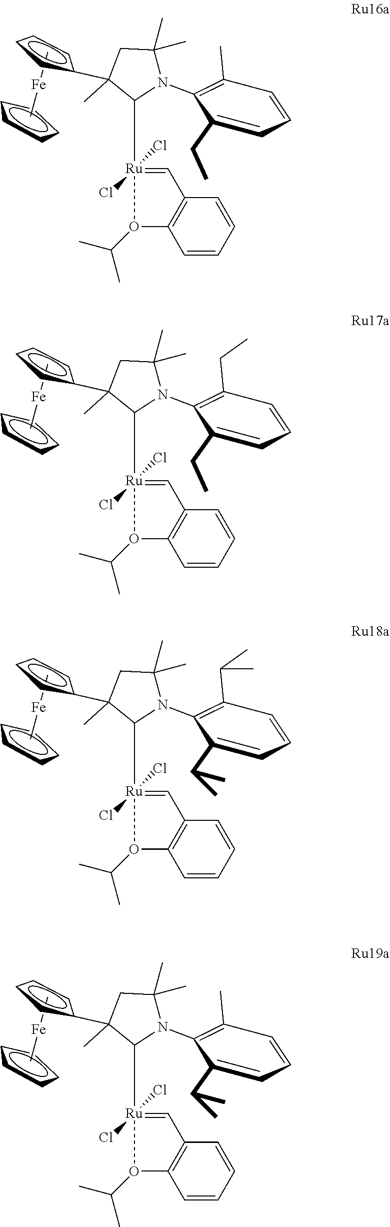

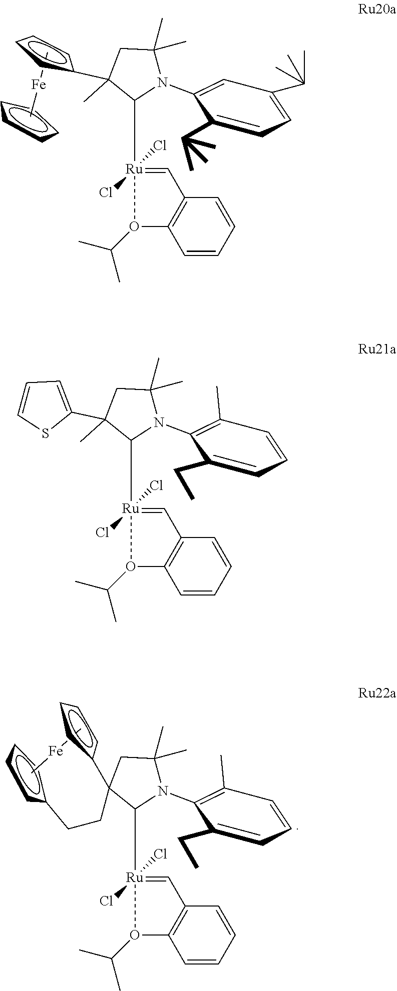

9. Method for the synthesis of the ruthenium complex of the formula 1a-Ru 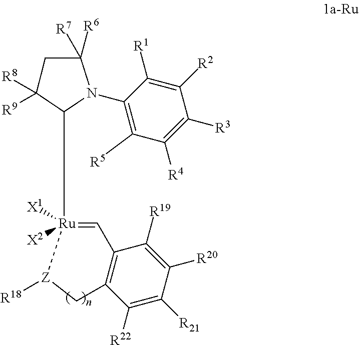
as defined in claim 6, characterized in that the alkylidene ruthenium complex of the formula 10 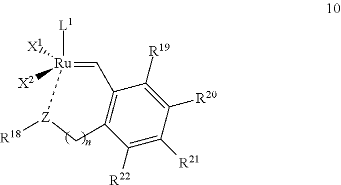
wherein:
L¹ denotes a neutral ligand selected from the group comprising pyridine or substituted pyridine, P(R′)₃, P(OR′)₃, O(R′)₂, N(R′)₃, wherein each R′ independently denotes C₁-C₁₂ alkyl, C₃-C₁₂ cycloalkyl, C₅-C₂₀ aryl, C₇-C₂₄ aralkyl, C₅-C₂₄ perfluoroaryl, a 5-12-membered heteroaryl;
N, Z, X¹, X² and the substituents R¹⁸, R¹⁹, R²⁰, R²¹ and R²² have the above defined meanings undergoes a reaction with the carbene of formula 8 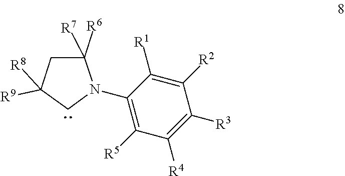
wherein the substituents R¹ to R⁹ have the above defined meanings.

10. Use of the compound of formula 1-Ru, as defined in claim 5, as a precatalyst and/or catalyst in olefin metathesis reactions, particularly in ring-closing metathesis (RCM), cross-metathesis (CM), homometathesis (cross-metathesis between two molecules of the same olefin), ethenolysis, isomerization, in a diastereoselective ring rearrangement metathesis (DRRM) reaction, alkene-alkyne (ene-yn) metathesis or ROMP or ADMET polymerization reaction.

11. Use according to claim 10, wherein the reaction is carried out in an organic solvent such as toluene, mesitylene, hexane, cyclohexane, ethyl acetate, methyl acetate, methyl carbonate, ethyl carbonate, tertbutyl methyl ether, cyclopentyl methyl ether, diethyl ether, THF, 2 Me-THF, 4-Me-THP, dioxane, DME, PAO. PEG, paraffin, esters of saturated fatty acids.

12. Use according to claim 10, wherein the reaction is carried out in a solvent-free system.

13. Use according to claim 10, wherein the reaction is carried out at a temperature between 20° C. and 200° C.

14. Use according to claim 10, wherein the reaction is carried out over a period between 5 minutes and 48 hours.

15. Use according to claim 10, wherein the compound 1-Ru is applied in amount not exceeding 10 mol %.

16. Use according to claim 10, wherein the compound 1-Ru is applied in amount of not exceeding 0.1 mol %.

17. Use according to claim 10, wherein the compound 1-Ru is added to the reaction mixture in solid portions and/or continuously using a pump as a solution in an organic solvent.

18. Use according to claim 10, wherein the gaseous reaction by-product, selected from ethylene, propylene, butylene, is actively removed from the reaction mixture using an inert gas barbotage or by means of a vacuum.
